(19) United States
(12) Patent Application Publication
(10) Pub. No.: US 2010/0317666 A1
(43) Pub. Date: Dec. 16, 2010

(54) Composition Comprising An NK-1 Receptor Antagonist And An SSRI For The Treatment Of Tinnitus And Hearing Loss

(75) Inventors: Soren Rahn Christensen, Cambridge (GB); Emilio Merlo Pich, Verona (IT); Emiliangelo Ratti, Verona (IT); Tadataka Yamada, Washington, WA (US)

Correspondence Address: GLAXOSMITHKLINE;GLOBAL PATENTS, FIVE MOORE DR., PO BOX 13398, MAIL STOP: C.2111F, RESEARCH TRIANGLE PARK, NC, 27709-3398, US

(73) Assignee: Glaxo Group Limited

(21) Appl. No.: 12/445,794

(22) PCT Filed: Oct. 18, 2007
(86) PCT No.: PCT/EP2007/061144

§ 371 Date: Jul. 15, 2009

(30) Foreign Application Priority Data
Oct. 20, 2006 (GB) 0621229.4

(51) Int. Cl. A61K 31/495; A61K 31/4525; A61K 31/496; A61K 31/4985; A61P 27/16

(52) U.S. Cl. 514/249; 514/321; 514/255.01; 514/253.13; 514/253.09

(57) ABSTRACT

The present invention relates to methods for treating a subject suffering from tinnitus, hearing loss, or tinnitus and hearing loss comprising administering to the subject an effective amount of an NK1 receptor antagonist alone, or in combination with an effective amount of a selective serotonin reuptake inhibitor. Compositions and pharmaceutical formulations are also provided.

The present invention relates to the new use of an NK1 receptor antagonist alone, or in combination with a selective serotonin reuptake inhibitor (SSRI) in the treatment of tinnitus and/or hearing loss.

Substance P is a short-chain polypeptide that functions as a neurotransmitter and a neuromodulator. It belongs to the tachykinin neuropeptide family. In the central nervous system Substance P has been associated with the regulation of mood disorders, anxiety, stress, reinforcement, neurogenesis, respiratory rhythm, neurotoxicity, nausea/emesis and pain. The endogenous receptor for Substance P is the neurokinin 1 receptor (NK1 receptor). A large number of NK1 receptor antagonists are known, including aprepitant (Emend™), which is marketed for use in the prevention of acute and delayed chemotherapy-induced nausea and vomiting and in the prevention of post operative nausea and vomiting. Other potential uses of NK1 receptor antagonists include treatment of anxiety and depression, pain, inflammatory dieases, over-active bladder, allergic disorders, CNS disorders, skin disorders, cough and gastrointestinal disorders.

Selective serotonin reuptake inhibitors (SSRIs) are a class of antidepressants used in the treatment of depression, anxiety and panic disorders, and personality disorders. They act via inhibition of the reuptake of the neurotransmitter serotonin (5-hydroxytryptamine) into the presynaptic cleft, increasing levels of serotonin within the synaptic cleft. There are several marketed SSRIs including citalopram, paroxetine, sertraline, fluoxetine and fluvoxamine.

U.S. Pat. No. 6,117,855 describes the use of a CNS-penetrant NK1 receptor antagonist together with an antidepressant or anti-anxiety drug for the manufacture of a medicament for the treatment or prevention of depression and/or anxiety.

WO2004/091624 (Glaxo Group Limited) relates to therapeutic combinations comprising paroxetine or physiologically acceptable salts or solvates thereof and 2-(S)-(4-fluoro-2-methyl-phenyl)-piperazine-1-carboxylic acid [1-(R)-(3,5-bis-trifluoromethyl-phenyl)-ethyl]-methyl-amide or physiologically acceptable salts or solvates thereof, to pharmaceutical compositions containing said combinations and their use in the treatment of depression and /or anxiety.

WO2004/004122 (Glaxo Group Limited) relates to therapeutic combinations comprising paroxetine or physiologically acceptable salts or solvates thereof and 4-(S)-(4-acetyl-piperazin-1-yl)-2-(R)-(4-fluoro-2-methyl-phenyl)-piperidine-1-carboxylic acid [1-(R)-(3,5-bis-trifluoromethyl-phenyl)-ethyl]-methylamide or pharmaceutically acceptable salts or solvates thereof, to pharmaceutical compositions containing said combinations and their use in the treatment of depression and /or anxiety.

WO2004/091615 (Glaxo Group Limited) relates to therapeutic combinations comprising paroxetine or physiologically acceptable salts or solvates thereof and 2-(R)-(4-fluoro-2-methyl-phenyl)-4-(S)-((8aS)-6-oxo-hexahydro-pyrrolo[1,2-a]-pyrazin-2-yl)-piperidine-1-carboxylic acid [1-(R)-(3,5-bis-trifluoromethyl-phenyl)-ethyl]-methylamide or pharmaceutically acceptable salts or solvates thereof, to pharmaceutical compositions containing said combinations and their use in the treatment of depression and/or anxiety.

Tinnitus, also known as ringing in the ears, has been defined as “the conscious expression of a sound that originates in an involuntary manner in the head of its owner”. This symptom occurs in about 10-15% of the general population, reaching disabling levels of severity in 0.5-1%. Tinnitus is a symptom with multiple aetiologies. It is commonly associated with sensorineural hearing loss, but may arise at many ignition points within the auditory pathway. Tinnitus, as an internally generated experience of sound, can be characterised by its pitch, loudness, timbre, and associated distress. About 40% of subjects with troublesome tinnitus also suffer from hyperacusis, a troublesome sensitivity and distress to everyday life sounds that would not trouble the majority of individuals.

Present treatments utilise counselling based therapy, the efficacy of which is difficult to determine using controlled trials. Uncertainty surrounding the mechanism of tinnitus generation makes it difficult to devise an effective treatment. The most consistent but transient pharmacological effect is produced by iv infusion of lidocaine, with up to 60% of subject responding with tinnitus partial or complete inhibition. Some evidence of improvement is reported when benzodiazepines, gabaergic drugs or tricyclic antidepressants where chronically administered. Modest effects have been reported after 8-12 weeks treatment with SSRIs, including high dose paroxetine (up to 50 mg/day). However, tinnitus associated to hearing loss remains an important unmet need as far as therapy is concerned.

Hearing loss and auditory processing problems can occur at any age, but in particular in adults or the elderly. According to the Royal National Institute for Deaf People (RNID), there are about nine million people who are deaf or hard of hearing in the UK. Most of them have lost their hearing gradually with increasing age (presbyacusis). Over half of all people aged over 60 are hard of hearing or deaf. One of the most common causes of hearing loss in adults is associated to acoustic trauma.

The solution provided by the present invention is use of an NK1 receptor antagonist alone, or in combination with a selective serotonin reuptake inhibitor (SSRI) for the treatment of tinnitus and/or hearing loss.

Thus, in a first aspect the invention provides the use of an NK1 receptor antagonist alone, or in combination with a selective serotonin reuptake inhibitor (SSRI) in the manufacture of a medicament for the treatment of tinnitus.

In a further aspect the invention provides the use of an NK1 receptor antagonist alone, or in combination with a selective serotonin reuptake inhibitor (SSRI) in the manufacture of a medicament for the treatment of hearing loss, or tinnitus and hearing loss.

In a further aspect the invention provides the use of an NK1 receptor antagonist in combination with a selective serotonin reuptake inhibitor (SSRI) in the manufacture of a medicament for the treatment of tinnitus, hearing loss, or tinnitus and hearing loss.

In another aspect the invention provides a method of treating a subject suffering from tinnitus which comprises administering to said subject an effective amount of an NK1 receptor antagonist alone, or in combination with a selective serotonin reuptake inhibitor (SSRI).

In another aspect the invention provides a method of treating a subject suffering from hearing loss, or tinnitus and hearing loss which comprises administering to said subject an effective amount of an NK1 receptor antagonist alone, or in combination with a selective serotonin reuptake inhibitor (SSRI).

In another aspect the invention provides a method of treating a subject suffering from tinnitus, hearing loss, or tinnitus and hearing loss which comprises administering to said subject an effective amount of an NK1 receptor antagonist in combination with a selective serotonin reuptake inhibitor (SSRI).

In another aspect the invention provides an NK1 receptor antagonist alone, or in combination with a selective serotonin reuptake inhibitor (SSRI) for use in the treatment of tinnitus.

In another aspect the invention provides an NK1 receptor antagonist alone, or in combination with a selective serotonin reuptake inhibitor (SSRI) for use in the treatment of hearing loss, or tinnitus and hearing loss.

In another aspect the invention provides an NK1 receptor antagonist in combination with a selective serotonin reuptake inhibitor (SSRI) for use in the treatment of tinnitus, hearing loss, or tinnitus and hearing loss.

In another aspect the invention provides a pharmaceutical composition comprising an NK1 receptor antagonist alone, or in combination with a selective serotonin reuptake inhibitor (SSRI) for use in the treatment of tinnitus.

In another aspect the invention provides a pharmaceutical formulation comprising an NK1 receptor antagonist alone, or in combination with a selective serotonin reuptake inhibitor (SSRI) for use in the treatment of hearing loss, or tinnitus and hearing loss.

In another aspect the invention provides a pharmaceutical formulation comprising an NK1 receptor antagonist in combination with a selective serotonin reuptake inhibitor (SSRI) for use in the treatment of tinnitus, hearing loss, or tinnitus and hearing loss.

In a further aspect the NK1 receptor antagonist for use in the present invention includes those generically and specifically disclosed in the following patent specifications whose disclosures are here incorporated by reference:

U.S. Pat. Specification Nos. 4,839,465, 5,338,845, 5,594,022, 6,169,097, 6,197,772, 6,222,038, 6,204,265, 6,329,392, 6,316,445, 2001039286, 2001034343, 2001029297, 2002193402, 2002147212, 2002147207, 2002143003 and 2002022624; and in European Patent Specification Nos. 284942, 327009, 333174, 336230, 360390, 394989, 428434, 429366, 436334, 443132, 446706, 482539, 484719, 499313, 512901, 512902, 514273, 514275, 517589, 520555, 522808, 525360, 528495, 532456, 533280, 577394, 591040, 615751, 684257, 1176144, 1110958, 1176144, 1172106, 1103545, and 1256578; and in International Patent Application Nos. 90/05525, 90/05729, 91/02745, 91/12266, 91/18016, 91/18899, 92/01688, 92/06079, 92/15585, 92/17449, 92/20676, 92/21677, 92/22569, 93/00331, 93/01159, 93/01160, 93/01165, 93/01169, 93/01170, 94/01402, 94/26735, 95/06645, 95/08549, 95/14017, 95/16679, 95/18124, 95/23798, 95/28389, 95/33744, 96/05181, 96/18643, 96/21661, 96/29326, 96/32386, 96/34857, 96/37489, 97/02824, 97/05110, 97/08166, 97/13514, 97/14671, 97/16440, 97/17362, 97/19074, 97/19084, 97/19942, 97/21702, 97/22597, 97/22604, 97/23455, 97/24324, 97/24350, 97/25322, 97/25988, 97/27185, 97/30989, 97/30990, 97/30991, 97/32865, 97/38692, 97/44035, 97/49393, 97/49710, 98/02158, 98/04561, 98/07694, 98/07722, 98/08826, 98/13369, 98/17276, 98/18761, 98/18785, 98/18788, 98/20010, 98/24438, 98/24439, 98/24440, 98/24441, 98/24442, 98/24442, 98/24443, 98/24444, 98/24445, 98/24446, 98/24447, 98/28297, 98/43639, 98/45262, 98/49170, 98/54187, 98/57954, 98/57972, 99/00388, 99/01444, 99/01451, 99/07677, 99/07681, 99/09987, 99/21823, 99/24423, 99/25364, 99/26924, 99/27938, 99/36424, 99/52903, 99/59583, 99/59972, 99/62893, 99/62900, 99/64000, 00/02859, 00/06544, 00/06571, 00/06572, 00/06578, 00/06580, 00/15621, 00/20003, 00/21512, 00/21564, 00/23061, 00/23062, 00/23066, 00/23072, 00/20389, 00/25745, 00/26214, 00/26215, 00/34243, 00/34274, 00/39114, 00/47562, 01/77069, 01/25233, 01/30348, 01/87866, 01/94346, 01/90083, 01/87838, 01/85732, 01/77100, 01/77089, 01/77069, 01/46176, 01/46167, 01/44200, 01/32625, 01/29027, 01/25219, 02/32865, 02/00631, 02/81461, 02/92604, 02/38575, 02/57250, 02/22574, 02/74771, 02/26710, 02/28853, 02/102372, 02/85458, 02/81457, 02/74771, 02/62784, 02/60898, 02/60875, 02/51848, 02/51807, 02/42280, 02/34699, 02/32867, 02/32866, 02/26724, 02/24673, 02/24629, 02/18346, 02/16344, 02/16343, 02/16324, 02/12168, 02/08232 and 02/06236; and in British Patent Specification Nos. 2216529, 2266529, 2268931, 2269170, 2269590, 2271774, 2292144, 2293168, 2293169 and 2302689; and in Japanese Patent Specification No 6040995.

In a further aspect the NK1 receptor antagonist for use in the present invention is selected from the group consisting of aprepitant (Emend™), netupitant (R-1124), fosaprepitant (MK-0517), SSR-240600, cizolirtine, AV 608, TA-5538, E 6039 and nolpitantium besilate (SR 140333).

In a further aspect the NK1 receptor antagonist for use in the present invention is a compound of formula (I)

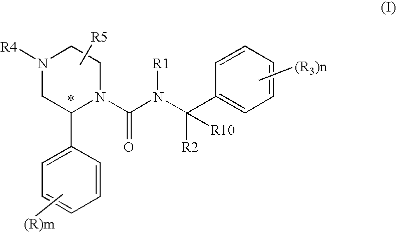

wherein
R represents a halogen atom or a C₁₋₄ alkyl group;
R1 represents hydrogen or a C₁₋₄alkyl group;
R2 represents hydrogen, a C₁₋₄alkyl, C₂₋₆ alkenyl or a C₃₋₇ cycloalkyl group; or R1 and R2 together with nitrogen and carbon atom to which they are attached respectively represent a 5-6 membered heterocyclic group;
R3 represents a trifluoromethyl, a C₁₋₄ alkyl, a C₁₋₄ alkoxy, a trifluoromethoxy or a halogen group;
R4 represents hydrogen, a (CH₂)_{q}R7 or a (CH₂)ᵣCO(CH₂)ₚR7 group;
R5 represents hydrogen, a C₁₋₄alkyl or a COR6 group;
R6 represents hydrogen, hydroxy, amino, methylamino, dimethylamino a 5 membered heteroaryl group containing 1 to 3 heteroatoms selected from oxygen, sulphur and nitrogen or a 6 membered heteroaryl group containing 1 to 3 nitrogen atoms;
R7 represents hydrogen, hydroxy or NR8R9 wherein R8 and R9 represent independently hydrogen or C₁₋₄ alkyl optionally substituted by hydroxy, or by amino;
R10 represents hydrogen, a C₁₋₄ alkyl group or
R10 together with R2 represents a a C₃₋₇ cycloalkyl group;
m is zero or an integer from 1 to 3; n is zero or an integer from 1 to 3; both p and r are independently zero or an integer from 1 to 4; q is an integer from 1 to 4; provided that, when R1 and R2 together with nitrogen and carbon atom to which they are attached respectively represent a 5 to 6 membered heterocyclic group, i) m is 1 or 2; ii) when m is 1, R is not fluorine and iii) when m is 2, the two substituents R are not both fluorine, and pharmaceutically acceptable salts, and solvates thereof.

Compounds of formula (I) and pharmaceutically acceptable salts and solvates thereof are described in PCT publication No. WO01/25219, published 12 Apr. 2001. The disclosure of this reference is incorporated herein in its entirety. Compounds of formula (I) and pharmaceutically acceptable salts and solvates thereof may be prepared by any method described in WO01/25219.

In a further aspect the NK1 receptor antagonist for use in the present invention is a compound of formula (II)

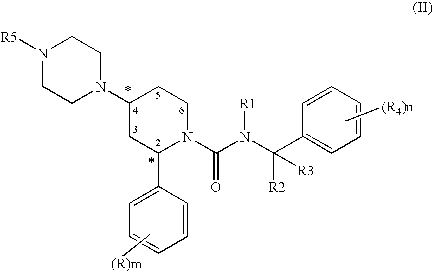

wherein
R represents a halogen atom or a C₁₋₄ alkyl group;
R₁ represents a C₁₋₄ alkyl group;
R₂ represents hydrogen or a C₁₋₄ alkyl group;
R₃ represents hydrogen or C₁₋₄ alkyl group;
R₄ represents a trifluoromethyl group;
R₅ represents hydrogen, a C₁₋₄ alkyl group or C(O)R₆;
R₆ represents C₁₋₄ alkyl, C₃₋₇ cycloalkyl, NH(C₁₋₄ alkyl) or N(C₁₋₄alkyl)₂;
m is zero or an integer from 1 to 3;
n is an integer from 1 to 3;
and pharmaceutically acceptable salts and solvates thereof.

Compounds of formula (II) and pharmaceutically acceptable salts and solvates thereof are described in PCT publication No. WO02/32867, published 25 Apr. 2002. The disclosure of this reference is incorporated herein in its entirety. Compounds of formula (II) and pharmaceutically acceptable salts and solvates thereof may be prepared by any method described in WO02/32867.

In a further aspect the NK1 receptor antagonist for use in the present invention is a compound of formula (III)

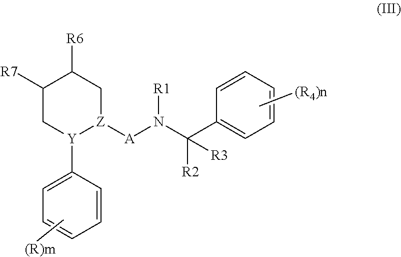

R represents halogen or C₁₋₄ alkyl;
R₁ represents C₁₋₄ alkyl;
R₂ or R₃ independently represent hydrogen or C₁₋₄ alkyl;
R₄ represents trifluoromethyl, C₁₋₄ alkyl, C₁₋₄ alkoxy, trifluoromethoxy or halogen;
R₅ represents hydrogen, C₁₋₄ alkyl or C₃₋₇ cycloalkyl;
R₆ is hydrogen and R₇ is a radical of formula (W):

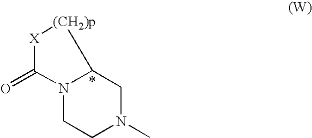

or R₆ is a radical of formula (W) and R₇ is hydrogen;
X represents CH₂, NR₅ or O;
Y represents Nitrogen and Z is CH or Y represents CH and Z is Nitrogen;
A represents C(O) or S(O)q, provided that when Y is nitrogen and Z is CH, A is not S(O)q;
m is zero or an integer from 1 to 3;
n is an integer from 1 to 3;
p and q are independently an integer from 1 to 2;
   and pharmaceutically acceptable salts and solvates thereof.

Compounds of formula (III) and pharmaceutically acceptable salts and solvates thereof are described in PCT publication No. WO 03/066635, published 14 Aug. 2003. The disclosure of this reference is incorporated herein in its entirety. Compounds of formula

(III) and pharmaceutically acceptable salts and solvates thereof may be prepared by any method described in WO 03/066635.

In a further aspect the NK1 receptor antagonist for use in the present invention is selected from the group consisting of 2-(S)-(4-fluoro-2-methyl-phenyl)-piperazine-1-carboxylic acid [1-(R)-(3,5-bis-trifluoromethyl-phenyl)-ethyl]-methyl-amide or pharmaceutically acceptable salts or solvates thereof, 4-(S)-(4-acetyl-piperazin-1-yl)-2-(R)-(4-fluoro-2-methyl-phenyl)-piperidine-1-carboxylic acid [1-(R)-(3,5-bis-trifluoromethyl-phenyl)-ethyl]-methylamide or pharmaceutically acceptable salts or solvates thereof, and 2-(R)-(4-fluoro-2-methyl-phenyl)-4-(S)-((8aS)-6-oxo-hexahydro-pyrrolo[1,2-a]-pyrazin-2-yl)-piperidine-1-carboxylic acid [1-(R)-(3,5-bis-trifluoromethyl-phenyl)-ethyl]-methylamide or pharmaceutically acceptable salts or solvates thereof.

Pharmaceutically salts and solvates as referred to hereinabove include acid addition salts formed with pharmaceutically acceptable organic or inorganic acids, for example hydrochlorides, hydrobromides, sulphates, alkyl- or arylsulphonates (e.g. methanesulphonates or p-toluenesulphonates), phosphates, acetates, citrates, succinates, tartrates, fumarates and maleates.

The solvates may, for example, be hydrates.

In a further aspect of the present invention the SSRI is a compound which functions as a selective serotonin reuptake inhibitor as determined using the standard pharmacological assay as set out in Wong, et al., Neuropsychopharmacology 8, 337-344 (1993), incorporated herein by reference. Many compounds have such activity, and no doubt many more will be identified in the future. In the practice of the present invention, it is intended to include reuptake inhibitors which show 50% effective concentrations of about 1000 nM or less, in the protocol described by Wong supra.

In a further aspect of the present invention the selective serotonin reuptake inhibitors of the present invention include, but are not limited to:

Fluoxetine, N-methyl-3-(p-trifluoromethylphenoxy)-3-phenylpropylamine, which is marketed in the hydrochloride salt form, and as the racemic mixture of its two enantiomers. U.S. Pat. No. 4,314,081 provides an early reference to this compound. Robertson et al., J. Med. Chem. 31, 1412 (1988), taught the separation of the R and S enantiomers of fluoxetine and showed that their activity as serotonin uptake inhibitors is similar to each other. In this document, the word “fluoxetine” will be used to mean any acid addition salt or the free base, and to include either the racemic mixture or either of the R and S enantiomers;

Citalopram, 1-[3-(dimethylamino)propyl]-1-(4-fluorophenyl)-1,3-dihydro-5-isobenzofuran-aqscarbonitrile, which is disclosed in U.S. Pat. No. 4,136,193 as a serotonin reuptake inhibitor. Its pharmacology was disclosed by Christensen et al., Eur. J. Pharmacol. 41, 153 (1977), and reports of its clinical effectiveness in depression may be found in Dufour et al., Int. Clin. Psychopharmacol. 2, 225 (1987), and Timmerman et al., ibid., 239;

Fluvoxamine, 5-methoxy-1-[4-(trifluoromethyl)-phenyl]-1-pentanone-O-(2-aminoethyl)-oxime, which is taught by U.S. Pat. No. 4,085,225. Scientific articles about the drug have been published by Claassen et al., Brit. J. Pharmacol. 60, 505 (1977); and De Wilde et al., J. Affective Disord. 4, 249 (1982); and Benfield et al., Drugs 32, 313 (1986);

Paroxetine, trans-(−)-3-[(1,3-benzodioxol-5-yloxy)methyl]-4-(4-fluorophenyl)piperidine, which may be found in U.S. Pat. Nos. 3,912,743 and 4,007,196. Reports of the drug's activity are in Lassen, Eur. J. Pharmacol. 47, 351 (1978); Hassan et al., Brit. J. Clin. Pharmacol. 19, 705 (1985); Laursen et al., Acta Psychiat. Scand. 71, 249 (1985); and Battegay et al., Neuropsychobiology 13, 31 (1985);. The term “paroxetine” as used herein includes any physiologically acceptable salts or solvates thereof. In particular, physiologically acceptable salts or solvates of paroxetine include, without limitation, paroxetine hydrochloride, paroxetine hydrochloride hemihydrate, paroxetine hydrochloride anhydrate, paroxetine mesylate and all polymorphic forms thereof.

Sertraline, (1S-cis)-4-(3,4-dichlorophenyl)-1,2,3,4-tetrahydro-N-methyl-1-naphthylamine hydrochloride, is a serotonin reuptake inhibitor which is marketed as an antidepressant. It is disclosed by U.S. Pat. No. 4,536,518;

All of the U.S. patents which have been mentioned above in connection with compounds used in the present invention are incorporated herein by reference.

In a further aspect the SSRI for use in the present invention is paroxetine.

In a further aspect the invention provides the use of 2-(S)-(4-fluoro-2-methyl-phenyl)-piperazine-1-carboxylic acid [1-(R)-(3,5-bis-trifluoromethyl-phenyl)-ethyl]methyl-amide or pharmaceutically acceptable salts or solvates thereof alone, or in combination with paroxetine in the manufacture of a medicament for the treatment of tinnitus.

In a further aspect the invention provides the use of 2-(S)-(4-fluoro-2-methyl-phenyl)-piperazine-1-carboxylic acid [1-(R)-(3,5-bis-trifluoromethyl-phenyl)-ethyl]methyl-amide or pharmaceutically acceptable salts or solvates thereof alone, or in combination with paroxetine in the manufacture of a medicament for the treatment of hearing loss, or tinnitus and hearing loss.

In a further aspect the invention provides the use of 2-(S)-(4-fluoro-2-methyl-phenyl)-piperazine-1-carboxylic acid [1-(R)-(3,5-bis-trifluoromethyl-phenyl)-ethyl]methyl-amide or pharmaceutically acceptable salts or solvates thereof in combination with paroxetine in the manufacture of a medicament for the treatment of tinnitus, hearing loss, or tinnitus and hearing loss.

In a further aspect the invention provides the use of 2-(S)-(4-fluoro-2-methyl-phenyl)-piperazine-1-carboxylic acid [1-(R)-(3,5-bis-trifluoromethyl-phenyl)-ethyl]-methyl-amide methanesulfonate or a solvate thereof in combination with paroxetine in the manufacture of a medicament for the treatment of tinnitus, hearing loss, or tinnitus and hearing loss.

In a further aspect the invention provides the use of vestipitant in combination with paroxetine in the manufacture of a medicament for the treatment of tinnitus, hearing loss, or tinnitus and hearing loss.

In a further aspect the invention provides a method of treating a subject suffering from tinnitus which comprises administering to said subject an effective amount of 2-(S)-(4-fluoro-2-methyl-phenyl)-piperazine-1-carboxylic acid [1-(R)-(3,5-bis-trifluoromethyl-phenyl)-ethyl]-methyl-amide or pharmaceutically acceptable salts or solvates thereof alone, or in combination with paroxetine.

In a further aspect the invention provides a method of treating a subject suffering from hearing loss, or tinnitus and hearing loss which comprises administering to said subject an effective amount of 2-(S)-(4-fluoro-2-methyl-phenyl)-piperazine-1-carboxylic acid [1-(R)-(3,5-bis-trifluoromethyl-phenyl)-ethyl]-methyl-amide or pharmaceutically acceptable salts or solvates thereof alone, or in combination with paroxetine.

In a further aspect the invention provides a method of treating a subject suffering from tinnitus, hearing loss, or tinnitus and hearing loss which comprises administering to said subject an effective amount of 2-(S)-(4-fluoro-2-methyl-phenyl)-piperazine-1-carboxylic acid [1-(R)-(3,5-bis-trifluoromethyl-phenyl)ethyl]-methyl-amide or pharmaceutically acceptable salts or solvates thereof in combination with paroxetine.

In another aspect the invention provides 2-(S)-(4-fluoro-2-methyl-phenyl)-piperazine-1-carboxylic acid [1-(R)-(3,5-bis-trifluoromethyl-phenyl)-ethyl]-methyl-amide or pharmaceutically acceptable salts or solvates thereof alone, or in combination with paroxetine for use in the treatment of tinnitus.

In another aspect the invention provides 2-(S)-(4-fluoro-2-methyl-phenyl)-piperazine-1-carboxylic acid [1-(R)-(3,5-bis-trifluoromethyl-phenyl)-ethyl]-methyl-amide or pharmaceutically acceptable salts or solvates thereof alone, or in combination with paroxetine for use in the treatment of hearing loss, or tinnitus and hearing loss.

In another aspect the invention provides 2-(S)-(4-fluoro-2-methyl-phenyl)-piperazine-1-carboxylic acid [1-(R)-(3,5-bis-trifluoromethyl-phenyl)-ethyl]-methyl-amide or pharmaceutically acceptable salts or solvates thereof in combination with paroxetine for use in the treatment of tinnitus, hearing loss, or tinnitus and hearing loss.

In another aspect the invention provides a pharmaceutical composition comprising 2-(S)-(4-fluoro-2-methyl-phenyl)-piperazine-1-carboxylic acid [1-(R)-(3,5-bis-trifluoromethyl-phenyl)-ethyl]-methyl-amide or pharmaceutically acceptable salts or solvates thereof alone, or in combination with paroxetine for use in the treatment of tinnitus.

In another aspect the invention provides a pharmaceutical formulation comprising 2-(S)-(4-fluoro-2-methyl-phenyl)-piperazine-1-carboxylic acid [1-(R)-(3,5-bis-trifluoromethyl-phenyl)-ethyl]-methyl-amide or pharmaceutically acceptable salts or solvates thereof alone, or in combination with paroxetine for use in the treatment of hearing loss, or tinnitus and hearing loss.

In another aspect the invention provides a pharmaceutical formulation comprising 2-(S)-(4-fluoro-2-methyl-phenyl)-piperazine-1-carboxylic acid [1-(R)-(3,5-bis-trifluoromethyl-phenyl)-ethyl]-methyl-amide or pharmaceutically acceptable salts or solvates thereof in combination with paroxetine for use in the treatment of tinnitus, hearing loss, or tinnitus and hearing loss.

In a further aspect the invention provides the use of 4-(S)-(4-acetyl-piperazin-1-yl)-2-(R)-(4-fluoro-2-methyl-phenyl)-piperidine-1-carboxylic acid [1-(R)-(3,5-bis-trifluoromethyl-phenyl)-ethyl]-methylamide or pharmaceutically acceptable salts or solvates thereof alone, or in combination with paroxetine in the manufacture of a medicament for the treatment of tinnitus.

In a further aspect the invention provides the use of 4-(S)-(4-acetyl-piperazin-1-yl)-2-(R)-(4-fluoro-2-methyl-phenyl)-piperidine-1-carboxylic acid [1-(R)-(3,5-bis-trifluoromethyl-phenyl)-ethyl]-methylamide or pharmaceutically acceptable salts or solvates thereof alone, or in combination with paroxetine in the manufacture of a medicament for the treatment of hearing loss, or tinnitus and hearing loss.

In a further aspect the invention provides the use of 4-(S)-(4-acetyl-piperazin-1-yl)-2-(R)-(4-fluoro-2-methyl-phenyl)-piperidine-1-carboxylic acid [1-(R)-(3,5-bis-trifluoromethyl-phenyl)-ethyl]-methylamide or pharmaceutically acceptable salts or solvates thereof in combination with paroxetine in the manufacture of a medicament for the treatment of tinnitus, hearing loss, or tinnitus and hearing loss.

In a further aspect the invention provides the use of casopitant in combination with paroxetine in the manufacture of a medicament for the treatment of tinnitus, hearing loss, or tinnitus and hearing loss.

In a further aspect the invention provides a method of treating a subject suffering from tinnitus which comprises administering to said subject an effective amount of 4-(S)-(4-acetyl-piperazin-1-yl)-2-(R)-(4-fluoro-2-methyl-phenyl)-piperidine-1-carboxylic acid [1-(R)-(3,5-bis-trifluoromethyl-phenyl)-ethyl]-methylamide or pharmaceutically acceptable salts or solvates thereof alone, or in combination with paroxetine.

In a further aspect the invention provides a method of treating a subject suffering from hearing loss, or tinnitus and hearing loss which comprises administering to said subject an effective amount of 4-(S)-(4-acetyl-piperazin-1-yl)-2-(R)-(4-fluoro-2-methyl-phenyl)-piperidine-1-carboxylic acid [1-(R)-(3,5-bis-trifluoromethyl-phenyl)-ethyl]-methylamide or pharmaceutically acceptable salts or solvates thereof alone, or in combination with paroxetine.

In a further aspect the invention provides a method of treating a subject suffering from tinnitus, hearing loss, or tinnitus and hearing loss which comprises administering to said subject an effective amount of 4-(S)-(4-acetyl-piperazin-1-yl)-2-(R)-(4-fluoro-2-methyl-phenyl)-piperidine-1-carboxylic acid [1-(R)-(3,5-bis-trifluoromethyl-phenyl)-ethyl]-methylamide or pharmaceutically acceptable salts or solvates thereof in combination with paroxetine.

In another aspect the invention provides 4-(S)-(4-acetyl-piperazin-1-yl)-2-(R)-(4-fluoro-2-methyl-phenyl)-piperidine-1-carboxylic acid [1-(R)-(3,5-bis-trifluoromethyl-phenyl)-ethyl]-methylamide or pharmaceutically acceptable salts or solvates thereof alone, or in combination with paroxetine for use in the treatment of tinnitus.

In another aspect the invention provides 4-(S)-(4-acetyl-piperazin-1-yl)-2-(R)-(4-fluoro-2-methyl-phenyl)-piperidine-1-carboxylic acid [1-(R)-(3,5-bis-trifluoromethyl-phenyl)-ethyl]-methylamide or pharmaceutically acceptable salts or solvates thereof alone, or in combination with paroxetine for use in the treatment of hearing loss, or tinnitus and hearing loss.

In another aspect the invention provides 4-(S)-(4-acetyl-piperazin-1-yl)-2-(R)-(4-fluoro-2-methyl-phenyl)-piperidine-1-carboxylic acid [1-(R)-(3,5-bis-trifluoromethyl-phenyl)-ethyl]-methylamide or pharmaceutically acceptable salts or solvates thereof in combination with paroxetine for use in the treatment of tinnitus, hearing loss, or tinnitus and hearing loss.

In another aspect the invention provides a pharmaceutical composition comprising 4-(S)-(4-acetyl-piperazin-1-yl)-2-(R)-(4-fluoro-2-methyl-phenyl)-piperidine-1-carboxylic acid [1-(R)-(3,5-bis-trifluoromethyl-phenyl)-ethyl]-methylamide or pharmaceutically acceptable salts or solvates thereof alone, or in combination with paroxetine for use in the treatment of tinnitus.

In another aspect the invention provides a pharmaceutical formulation comprising 4-(S)-(4-acetyl-piperazin-1-yl)-2-(R)-(4-fluoro-2-methyl-phenyl)-piperidine-1-carboxylic acid [1-(R)-(3,5-bis-trifluoromethyl-phenyl)-ethyl]-methylamide or pharmaceutically acceptable salts or solvates thereof alone, or in combination with paroxetine for use in the treatment of hearing loss, or tinnitus and hearing loss.

In another aspect the invention provides a pharmaceutical formulation comprising 4-(S)-(4-acetyl-piperazin-1-yl)-2-(R)-(4-fluoro-2-methyl-phenyl)-piperidine-1-carboxylic acid [1-(R)-(3,5-bis-trifluoromethyl-phenyl)-ethyl]-methylamide or pharmaceutically acceptable salts or solvates thereof in combination with paroxetine for use in the treatment of tinnitus, hearing loss, or tinnitus and hearing loss.

In a further aspect the invention provides the use of 2-(R)-(4-fluoro-2-methyl-phenyl)-4-(S)-((8aS)-6-oxo-hexahydro-pyrrolo[1,2-a]-pyrazin-2-yl)-piperidine-1-carboxylic acid [1-(R)-(3,5-bis-trifluoromethyl-phenyl)-ethyl]-methylamide or pharmaceutically acceptable salts or solvates thereof alone, or in combination with paroxetine in the manufacture of a medicament for the treatment of tinnitus.

In a further aspect the invention provides the use of 2-(R)-(4-fluoro-2-methyl-phenyl)-4-(S)-((8aS)-6-oxo-hexahydro-pyrrolo[1,2-a]-pyrazin-2-yl)-piperidine-1-carboxylic acid [1-(R)-(3,5-bis-trifluoromethyl-phenyl)-ethyl]-methylamide or pharmaceutically acceptable salts or solvates thereof alone, or in combination with paroxetine in the manufacture of a medicament for the treatment of hearing loss, or tinnitus and hearing loss.

In a further aspect the invention provides the use of 2-(R)-(4-fluoro-2-methyl-phenyl)-4-(S)-((8aS)-6-oxo-hexahydro-pyrrolo[1,2-a]-pyrazin-2-yl)-piperidine-1-carboxylic acid [1-(R)-(3,5-bis-trifluoromethyl-phenyl)-ethyl]-methylamide or pharmaceutically acceptable salts or solvates thereof in combination with paroxetine in the manufacture of a medicament for the treatment of tinnitus, hearing loss, or tinnitus and hearing loss.

In a further aspect the invention provides the use of orvepitant in combination with paroxetine in the manufacture of a medicament for the treatment of tinnitus, hearing loss, or tinnitus and hearing loss.

In a further aspect the invention provides a method of treating a subject suffering from tinnitus which comprises administering to said subject an effective amount of 2-(R)-(4-fluoro-2-methyl-phenyl)-4-(S)-((8aS)-6-oxo-hexahydro-pyrrolo[1,2-a]-pyrazin-2-yl)-piperidine-1-carboxylic acid [1-(R)-(3,5-bis-trifluoromethyl-phenyl)ethyl]-methylamide or pharmaceutically acceptable salts or solvates thereof alone, or in combination with paroxetine.

In a further aspect the invention provides a method of treating a subject suffering from hearing loss, or tinnitus and hearing loss which comprises administering to said subject an effective amount of 2-(R)-(4-fluoro-2-methyl-phenyl)-4-(S)-((8aS)-6-oxo-hexahydro-pyrrolo[1,2-a]-pyrazin-2-yl)-piperidine-1-carboxylic acid [1-(R)-(3,5-bis-trifluoromethyl-phenyl)-ethyl]-methylamide or pharmaceutically acceptable salts or solvates thereof alone, or in combination with paroxetine.

In a further aspect the invention provides a method of treating a subject suffering from tinnitus, hearing loss, or tinnitus and hearing loss which comprises administering to said subject an effective amount of 2-(R)-(4-fluoro-2-methyl-phenyl)-4-(S)-((8aS)-6-oxo-hexahydro-pyrrolo[1,2-a]-pyrazin-2-yl)-piperidine-1-carboxylic acid [1-(R)-(3,5-bis-trifluoromethyl-phenyl)-ethyl]-methylamide or pharmaceutically acceptable salts or solvates thereof in combination with paroxetine.

In another aspect the invention provides 2-(R)-(4-fluoro-2-methyl-phenyl)-4-(S)-((8aS)-6-oxo-hexahydro-pyrrolo[1,2-a]-pyrazin-2-yl)-piperidine-1-carboxylic acid [1-(R)-(3,5-bis-trifluoromethyl-phenyl)-ethyl]-methylamide or pharmaceutically acceptable salts or solvates thereof alone, or in combination with paroxetine for use in the treatment of tinnitus.

In another aspect the invention provides 2-(R)-(4-fluoro-2-methyl-phenyl)-4-(S)-((8aS)-6-oxo-hexahydro-pyrrolo[1,2-a]-pyrazin-2-yl)-piperidine-1-carboxylic acid [1-(R)-(3,5-bis-trifluoromethyl-phenyl)-ethyl]-methylamide or pharmaceutically acceptable salts or solvates thereof alone, or in combination with paroxetine for use in the treatment of hearing loss, or tinnitus and hearing loss.

In another aspect the invention provides 2-(R)-(4-fluoro-2-methyl-phenyl)-4-(S)-((8aS)-6-oxo-hexahydro-pyrrolo[1,2-a]-pyrazin-2-yl)-piperidine-1-carboxylic acid [1-(R)-(3,5-bis-trifluoromethyl-phenyl)-ethyl]-methylamide or pharmaceutically acceptable salts or solvates thereof in combination with paroxetine for use in the treatment of tinnitus, hearing loss, or tinnitus and hearing loss.

In another aspect the invention provides a pharmaceutical composition 2-(R)-(4-fluoro-2-methyl-phenyl)-4-(S)-((8aS)-6-oxo-hexahydro-pyrrolo[1,2-a]-pyrazin-2-yl)-piperidine-1-carboxylic acid [1-(R)-(3,5-bis-trifluoromethyl-phenyl)-ethyl]-methylamide or pharmaceutically acceptable salts or solvates thereof alone, or in combination with paroxetine for use in the treatment of tinnitus.

In another aspect the invention provides a pharmaceutical formulation 2-(R)-(4-fluoro-2-methyl-phenyl)-4-(S)-((8aS)-6-oxo-hexahydro-pyrrolo[1,2-a]-pyrazin-2-yl)-piperidine-1-carboxylic acid [1-(R)-(3,5-bis-trifluoromethyl-phenyl)-ethyl]methylamide or pharmaceutically acceptable salts or solvates thereof alone, or in combination with paroxetine for use in the treatment of hearing loss, or tinnitus and hearing loss.

In another aspect the invention provides a pharmaceutical formulation 2-(R)-(4-fluoro-2-methyl-phenyl)-4-(S)-((8aS)-6-oxo-hexahydro-pyrrolo[1,2-a]-pyrazin-2-yl)-piperidine-1-carboxylic acid [1-(R)-(3,5-bis-trifluoromethyl-phenyl)-ethyl]-methylamide or pharmaceutically acceptable salts or solvates thereof in combination with paroxetine for use in the treatment of tinnitus, hearing loss, or tinnitus and hearing loss.

The adjunctive therapy of the present invention is carried out by administering an NK1 receptor antagonist together with an SSRI in any manner which provides effective levels of the compounds in the body at the same time. It will be appreciated that the compounds of the combination may be administered simultaneously, either in the same or separate dosage forms, or sequentially. It will also be understood that the compounds of the combination or the physiologically acceptable salts or solvates thereof, whether presented simultaneously or sequentially, may be administered individually, or in multiples, or in any combination thereof.

The ratio of the NK1 receptor anatgonist to the SSRI, in the combination according to the invention may be for example, from 1:15 to 10: 1 (measured by weight of the free bases). In another aspect the ratio may be from 1:4 to 4:1 (measured by weight of the free bases). In a further aspect the ratio may be from 1:4 to 1:1 (measured by weight of the free bases).

The amount of a combination according to the invention required to be effective as a treatment for tinnitus may, of course, vary and is ultimately at the discretion of the medical practitioner. The factors to be considered include the route of administration and nature of the formulation, the subject mammal's body weight, age and general condition and the nature and severity of the condition to be treated.

Unless otherwise indicated, all weights of active ingredients are calculated in terms of the drug per se. The desired dose may preferably be presented as one, two, three, four, five, six or more sub-doses administered at appropriate intervals throughout the day.

While it is possible for the active ingredients of the combination to be administered as the raw chemical, it is preferable to present them as a pharmaceutical formulation.

Pharmaceutical formulations according to the present invention comprise a combination according to the invention together with one or more pharmaceutically acceptable carriers or excipients and optionally other therapeutic agents. The carrier(s) must be acceptable in the sense of being compatible with the other ingredients of the formula and not deleterious to the recipient thereof. When the individual components of the combination are administered separately, they are generally each presented as a pharmaceutical formulation. The references hereinafter to formulations refer, unless otherwise stated, to formulations containing either the combination or a component thereof.

A combination of an NK1 receptor antagonist and an SSRI for use in the treatment of tinnitus may conveniently be presented as a pharmaceutical formulation in a unitary dosage form. Thus pharmaceutical formulations incorporating both compounds are important embodiments of the present invention. Such formulations may take any physical form which is pharmaceutically acceptable, for example orally usable pharmaceutical formulations. Such adjunctive pharmaceutical formulations contain an effective amount of each of the compounds, which effective amount is related to the daily dose of the compounds to be administered. Each adjunctive dosage unit may contain the daily doses of all compounds, or may contain a fraction of the daily doses, such as one-third of the doses. Alternatively, each dosage unit may contain the entire dose of one of the compounds, and a fraction of the dose of the other compounds. In such case, the patient would daily take one of the combination dosage units, and one or more units containing only the other compound. The amounts of each drug to be contained in each dosage unit may depend on the identity of the drugs chosen for the therapy.

Formulations of the NK1 receptor antagonist and the SSRI for use in the present invention include those suitable for oral, rectal, nasal, topical (including transdermal, buccal and sublingual), vaginal or parenteral (including subcutaneous, intramuscular, intravenous and intradermal) administration. The formulations may conveniently be presented in unit dosage form and may be prepared by any methods well known in the art of pharmacy. Such methods represent a further feature of the present invention and include the step of bringing into association the active ingredients with the carrier which constitutes one or more accessory ingredients. In general, the formulations are prepared by uniformly and intimately bringing into association the active ingredients with liquid carriers or finely divided solid carriers or both, and then if necessary shaping the product.

Formulations of the present invention suitable for oral administration may be presented as discrete units such as capsules, caplets, cachets or tablets each containing a predetermined amount of the active ingredients; as a powder or granules; as a solution or a suspension in an aqueous or non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredients in a free-flowing form such as a powder or granules, optionally mixed with a binder (e.g. povidone, gelatin, hydroxypropylmethyl cellulose), lubricant, inert diluent, preservative, disintegrant (e.g. sodium starch glycollate, sodium croscarmellose cross-linked povidone, cross-linked sodium carboxymethyl cellulose) surface-active or dispersing agent. Moulded tablets may be made by moulding a mixture of the powdered compound moistened with an inert liquid diluent in a suitable machine. The tablets may optionally be coated or scored and may be formulated so as to provide slow or controlled release of the active ingredients therein using, for example, hydroxypropylmethyl cellulose in varying proportions to provide the desired release profile. Tablets may optionally be provided with an enteric coating, to provide release in parts of the gut other than the stomach.

Formulations suitable for topical administration in the mouth include lozenges comprising the active ingredients in a flavored base, usually sucrose and acacia or tragacanth; pastilles comprising the active ingredients in an inert basis such as gelatin and glycerin, or sucrose and acacia; and mouthwashes comprising the active ingredients in a suitable liquid carrier. Formulations for rectal administration may be presented as a suppository with a suitable base comprising, for example, cocoa butter or polyethylene glycols.

Topical administration may also be by means of a transdermal iontophoretic device.

Formulations suitable for vaginal administration may be presented as tablets, pessaries, tampons, creams, gels, pastes, foams or spray formulations containing in addition to the active ingredients such carriers as are known in the art to be appropriate.

Pharmaceutical formulations suitable for rectal administration wherein the carrier is a solid are most preferably presented as unit dose suppositories. Suitable carriers include cocoa butter and other materials commonly used in the art. The suppositories may be conveniently formed by admixture of the active combination with the softened or melted carrier(s) followed by chilling and shaping in molds.

Formulations suitable for parenteral administration include aqueous and nonaqueous isotonic sterile injection solutions which may contain anti-oxidants, buffers, preservatives and solutes which render the formulation isotonic with the blood of the intended recipient; and aqueous and non-aqueous sterile suspensions which may include suspending agents and thickening agents; and liposomes or other microparticulate systems which are designed to target the compound to blood components or one or more organs. The formulations may be presented in unit-dose or multi-dose sealed containers, for example, ampoules and vials, and may be stored in a freeze-dried (lyophilized) condition requiring only the addition of the sterile liquid carrier, for example water for injection, immediately prior to use. Extemporaneous injection solutions and suspensions may be prepared from sterile powders, granules and tablets of the kind previously described.

It should be understood that in addition to the ingredients particularly mentioned above the formulations of this invention may include other agents conventional in the art having regard to the type of formulation in question, for example, those suitable for oral administration may include such further agents as sweeteners, thickeners and flavoring agents.

The pharmaceutical formulation of the invention containing the two active ingredients may be prepared according to conventional techniques well known in the pharmaceutical industry. Thus, for example paroxetine and 2-(S)-(4-Fluoro-2-methyl-phenyl)-piperazine-1-carboxylic acid [1-(R)-(3,5-bis-trifluoromethyl-phenyl)-ethyl]-methyl-amide or physiologically acceptable salts or solvates thereof, may be admixed together with suitable excipients such as those described above for the formulation of each of the active ingredients separately. Tablets may be prepared, for example by direct compression of such a mixture or using other conventional methods. Bilayer tablets may be prepared according to conventional procedure. Thus, for example, by separately compressing the two blends in a suitable tabletting machine with two filling stations. Capsules may be prepared by filling the blend along with suitable excipients into gelatin capsules, using a suitable filling machine. Controlled release forms for oral or rectal administration may be formulated in a conventional manner associated with controlled release forms.

Pharmaceutical formulations are often prescribed to the patient in “patient packs” containing the whole course of treatment in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in traditional prescriptions. The inclusion of a package insert has been shown to improve patient compliance with the physician's instructions and, therefore, lead generally to more successful treatment.

It will be understood that the administration of the combination of the invention by means of a single patient pack, or patient packs of each formulation, containing within a package insert instructing the patient to the correct use of the invention is a desirable additional feature of this invention.

According to a further aspect of the invention provided is a multiple, for example, double or triple, pack comprising an NK1 receptor antagonist and an SSRI, and an information insert containing directions on the use of the combination of the invention.

### EXPERIMENTAL DATA

### Intermediate 1

### (4-Fluoro-2-methyl-phenyl)-oxo-acetic acid methyl ester

1) To a suspension of magnesium turnings (617 mg) in anh. THF (6 mL), at r.t., under N2, a small crystal of I2 was added, followed by 10% of a solution of commercial 2-bromo-5-fluorotoluene (4.0 g) in anh. THF (15 mL). The suspension was heated gently (heat gun) until the brown colour disappeared. The remaining bromide solution was added drop-wise, maintaining the reaction mixture warm (50-60° C.) with an oil bath. After the addition was complete (15 min), the suspension was stirred at 70° C. until the magnesium turnings had almost completely reacted (2 hr). The new brown solution was used in the next step.

2) A solution of LiBr (4.41 g) in anh. THF (50 mL) was added drop-wise to a suspension of CuBr (3.64 g) in anh. THF (50 mL). The reaction mixture was stirred at r.t. for 1 hr (dark green solution with a small amount of white solid in suspension). The Grignard solution previously prepared was then added dropwise (an ice bath was used to maintain the temperature <25° C.) followed by methyl oxalyl chloride (1.95 mL). The reaction mixture was stirred at r.t. for 2 hr. The THF was evaporated and the residue was taken up in AcOEt. The organic layer was washed with sat.aq. NH4Cl (2×) and dried. The solids were filtered and the solvent evaporated to give a crude oil, which was purified by flash chromatography (CH/AcOEt 95:5) to obtain the title compound as a clear oil (2.44 g). NMR (CDCl3): δ (ppm) 7.74 (m, 1H), 6.98-7.04 (m, 2H), 3.96 (s, 3H), 2.61 (s, 3H).

### Intermediate 2

### 3-(4-Fluoro-2-methyl-phenyl)-5,6-dihydro-1H-pyrazin-2-one

To a solution of intermediate 1 (2.01 g) and ethylenediamine (684 μL) in toluene (40 mL), at r.t., under N2, anh. Na2SO4 (2 g) was added. The reaction mixture was heated at reflux for 6 hr. It was then cooled down to r.t. and filtered. The solids were rinsed with DCM. The solvent was evaporated and the crude oil was purified by flash chromatography (AcOEt) affording the title compound as a white solid (1.29 g).

NMR (CDCl3): δ (ppm) 7.33 (m, 1H), 6.95-6.90 (m, 2H), 6.56 (m, 1H), 3.97 (m, 2H), 3.58 (m, 2H), 2.31 (s, 3H).

### Intermediate 2a

### 3-(4-Fluoro-2-methyl-phenyl)-piperazin-2-one

To a solution, at 25° C., of intermediate 2 (168 g) in Methanol (2400 mL) under nitrogen, Pd/C 10% (44 g) was added. The reaction mixture was placed under a H₂ atmosphere and stirred at 25° C. for about 16 hours (till no further hydrogen was consumed and the reaction was completed by TLC, EA/MeOH 9/1). The catalyst was filtered in nitrogen atmosphere and the solvent was removed to low volume (360 mL) then Methanol (2040 mL) and Ethyl Acetate (9600 mL) were added and a silica pad (800 g) was performed; the eluted solution was concentrated to obtain the title compound (168 g).

¹H-NMR (DMSO) δ (ppm) 7.77 (bm, 1H); 7.24 (dd, 1H); 6.96 (dd, 1H); 6.92 (td, 1H); 4.43 (s, 1H); 3.30 (m, 1H); 3.14 (m, 1H); 2.92 (m, 1H); 2.82 (m, 2H); 2.33 (s, 3H).

### Intermediate 3

### (+)(S)-3-(4-Fluoro-2-methyl-phenyl)-piperazin-2-one

### Method A

To a suspension of intermediate 2 (35 g) in AcOEt (900 mL), L(+)-Mandelic Acid (27.3 g) was added. The suspension was stirred at r.t. for 1 hr then at 3-5° C. for 2 hr, filtered and dried under vacuum at r.t to obtain crude L(+)-mandelate 3-(4-fluoro-2-methyl-phenyl)-piperazin-2-one (37 g), which was suspended in AcOEt (370 mL) and heated to reflux till complete solubilisation then cooled to room temperature and stirred for further 2 hours, filtered, washed with of AcOEt (150 mL) and dried under vacuum obtaining (+) L-mandelate 3-(4-Fluoro-2-methyl-phenyl)-5,6 pyrazin-2-one (30.4 g) as white solid. This material (30.4 g) was suspended in AcOEt (300 mL) and treated with NaOH (0.73M, 155 mL) saturated with NaCl. The organic phase was then washed with water (90 mL). The aqueous phase was counter-extracted 4 times with AcOEt (90 mL). The combined organic phase (1800 mL) was dried on 10 g of Na2SO4 and concentrated under vacuum obtaining the title compound (25.04 g) as white foam.

### Method B

To a solution, heated at 45° C., of intermediate 2a (168 g) in Ethyl Acetate (2000 mL) L(+)-Mandelic Acid (116 g) and 3,5-dichloro-salicilaldehyde (10.8 g) were added. The solution was stirred for 30min at 45° C. then seeded with white crystals of L(+)mandelate-3-(4-Fluoro-2-methyl-phenyl)-piperazin-2-one (0.4 g). The obtained suspension was stirred under nitrogen atmosphere at 45° C. for 16 hours then stirred for further 4 hour at 0° C., washed with cooled Ethyl Acetate (2×200 ml) then dried under vacuum at room temperature for 2 hours to obtain L(+)mandelate-3-(4-Fluoro-2-methyl-phenyl)-piperazin-2-one(126.22 g) as a white/yellowish solid which was suspended in DCM (2760 mL) then NaOH 0.8M in Brine (17.35 g of NaOH in 530 mL of Brine) was added. The organic phase was then washed with Brine (380 mL) and the aqueous phase was counter extracted four times with DCM (4×1500 mL). The combined organic phase was dried and concentrated to obtain the title compound (60.35 g).

¹H-NMR (DMSO) δ (ppm) 7.77 (bm, 1H); 7.24 (dd, 1H); 6.96 (dd, 1H); 6.92 (td, 1H); 4.43 (s, 1H); 3.30 (m, 1H); 3.14 (m, 1H); 2.92 (m, 1H); 2.82 (m, 2H); 2.33 (s, 3H).

HPLC:Chiralcel OJ (4.6×250 mm) from Daicel; Mobile Phase:n-Hexane/Ethanol 80:20 v/v;Flow:1 mL/min; Detector:UV @ 265 nm (or 210 nm for higher signals); Dissolution phase:n-Hexane/Ethanol 80/20 v/v;

Sample Concentration 1 mg/ml; Injection:5 uL; Retention times: 2:8.4 min. [α]_{D}(solvent CHCl3, Source: Na; Cell volume [ml]: 1; Cell pathlength [dm]:1; Cell temperature [° C.]:20; Wavelength [nm]: 589; Conc. sample [% p/v]:1.17)=+17.9.

### Intermediate 4

### (S)-3-(4-Fluoro-2-methyl-phenyl)-piperazine dihydrochloride

To a solution of intermediate 3 (60.35 g) in dry THF (180 ml), at 0-3° C., under N₂, BH₃. THF 1M/THF (1220 mL) was added dropwise. The solution was refluxed for 4 hours then cooled to 0-3° C. and methanol (240 mL) was added. The reaction mixture was heated to room temperature then it was concentrated to dryness. The residue was redissolved in methanol (603.5 mL), excess HCl 1N in Et₂O (1207 mL) was added and the mixture was refluxed for 2 hours then cooled at 3° C. for 4 hours. The suspension was filtered to obtain a white solid that was washed with Et₂O (60.35 mL) and dried to yield the title compound (72.02 g)

¹H-NMR (DMSO) δ (ppm) 11.0-9.5 (b, 4H); 7.99-7.19 (dd-m, 3H); 4.96 (dd, 1H); 3.65-3.15 (m, 6H); 2.42 (s, 3H).

### Intermediate 5

### (R)-3,5-bis-trifluoromethyl-phenyl)-ethyl]-methyl-amine

To a solution of 3,5-bis-trifluoromethylacetophenone (300 g) in MeOH (1120 mL), a solution of methylamine 8M in EtOH (372 mL) was added dropwise during 15 min. at 25° C. under N₂. The mixture was stirred for 24 hrs at 25° C. under N₂. Then, NaBH₄ was portionwise added over 30 min (27.9 g) at 0° C. A second amount of NaBH₄ was added over 30 min (17.1 g) and the mixture stirred for further 1.5 hrs.

The mixture was concentrated by evaporating 600 mL of solvent under vacuum then it was slowly poured into a mixture of AcOEt (1500 mL)/NH₄Cl sat (750 mL) and water (750 mL). The water phase was back-extracted with AcOEt (1500 mL). The combined organic phases were washed with Water/Brine (150 mL/150 mL) then evaporated to obtain 3,5-bis-trifluoromethyl-phenyl)-ethyl]-methyl-amine (305 g) as a yellow oil

To a solution of 3,5-bis-trifluoromethyl-phenyl)-ethyl]-methyl-amine (245.6 g) in EtOAc (2380 mL), L(+) malic acid was added portionwise (118 g). The suspension was stirred for 2 hrs at 25° C. then 3 hrs at 0° C. The suspension was filtered and the cake was washed with EtOAc (240 mL). The solid was dried under vacuum obtaining crude L(+)malate3,5-bis-trifluoromethyl-phenyl)-ethyl]-methyl-amine (135.3 g) as a white solid which was suspended in Ethyl acetate (1760 mL) then heated to reflux till complete dissolution and then cooled at 25° C. The suspension was filtered, washed with Ethyl acetate (135 mL) then dried to obtain L(+)malate3,5-bis-trifluoromethyl-phenyl)-ethyl]-methyl-amine (128.5 g). The solid was stirred in a mixture of NaOH 10% v/v (720 mL) and Ethyl acetate (650 mL). Organic phase was washed with water (720 mL), then concentrated to yield the title compound (82.2 g).

¹H-NMR (DMSO) δ (ppm) 7.99 (s, 2H); 7.85 (s, 1H); 3.78 (q, 1H); 2.34 (s, 1H); 2.09 (s, 3H); 1.23 (d, 3H).

### REPRESENTATIVE EXAMPLE 1

### 2-(S)-(4-Fluoro-2-methyl-phenyl)-piperazine-1-carboxylic acid [1-(R)-(3,5-bis-trifluoromethyl-phenyl)-ethyl]-methyl-amide methansulphonate

To a suspension of intermediate 4 (4.9 Kg) in AcOEt (137.2 L), triethylamine (5.63 L) was added. The mixture was cooled to 0° C. then a solution of diterbuthyl dicarbonate (3.134 Kg) in AcOEt (24.5 L) was added in 35 min, maintaining the temperature between 0 and 5° C. The suspension was stirred at 0° C. for 15 min, at 20/25° C. for 1 hr, then washed with water (3×39.2 L), concentrated to 24.5 L and then added to a solution of triphosgene (1.97 Kg) in AcOEt (24.5 L) cooled to 0° C. Triethylamine (3.28 L) was then added in 40 min, maintaining the temperature between 0 and 8° C. The suspension was stirred for 1 h and 45 min at 20/25° C. and 30 min at 70 C and then the solution of intermediate 5 diluted with AcOEt (49 L) and triethylamine (2.6 L) was added in 30 min. The mixture was refluxed for 15 hrs.

The reaction mixture, cooled at 20/25° C. was treated with aqueous solution of NaOH 10% v/v (36.75 L). Organic phase was washed with HCl 4% v/v (46.55 L) and NaCl 11,5% p/p (4×24.5 L) then concentrated to 14.7 L. and diluted with Ciclohexane (39.2 L). The mixture was filtered through a silica pad (4.9 Kg) that was washed twice with a mixture of CH/AcOEt 85/15 (2×49 L). To the Eluted phases (14.7 L) cooled at 20/25° C., methyl tertbutyl ether (49 L) and methansulphonic acid (4.067 L) were added. The mixture was washed with NaOH 10% v/v (31.85 L) then with water (4×31.85 L). Organic phase was concentrated to 9.8 L, methyl tertbutyl ether (49 L) was added and the solution filtered through a 5 micron filter then concentrated to 9.8 L. At 20/25° C. MTBE (29.4 L) and metansulphonic acid (1.098 L) were added. The suspension was refluxed for 10 min, stirred at 20/25° C. for 10 hrs and 2 hrs at O° C. Then the precipitate was filtered, washed with methyl tertbutyl ether (4.9 L) dried under vacuum at 20/25° C. for 24 hrs to obtain the title compound (5.519 Kg.) as white solid.

¹H-NMR (DMSO) δ (ppm) 8.99 (bm, 1H); 8.66 (bm, 1H); 8.00 (bs, 1H); 7.69 (bs, 2H); 7.27 (dd, 1H); 7.00 (dd, 1H); 6.83 (m, 1H); 5.32 (q, 1H); 4.47 (dd, 1H); 3.50-3.20 (m, 4H); 2.96 (m, 2H); 2.72 (s, 3H); 2.37 (s, 3H); 2.28 (s, 3H); 1.46 (d, 3H).

ES⁺: m/z 492 [MH—CH₃SO₃H]⁺

ES⁻: m/z 586 [M−H]⁻; 95 [CH₃SO₃]⁻

### Intermediate 6

### 1-(Benzyloxycarbonyl)-2-(4-fluoro-2-methyl-phenyl)-2,3-dihydro-4-pyridone

A small amount of iodine was added to a suspension of magnesium turnings (13.2 g) in dry THF (300 mL), at r.t., under a nitrogen atmosphere, then the mixture was vigorously refluxed for 20 minutes. To this suspension, a 15% of a solution of 2-bromo-5-fluoro-toluene (52.5 mL) in anhydrous THF (300 mL) was added. The suspension was heated under vigorous reflux until the brown colour disappeared. The remaining part of the bromide solution was added drop-wise over 1 hour to the refluxing suspension which was then stirred for a further 1 hour. This solution of Grignard reagent was then added drop-wise to the pyridinium salt obtained from benzyl chloroformate (48.7 mL) and 4-methoxypyridine (25 mL) in dry THF (900 mL) at −23° C.

The obtained solution was stirred 1 hour at −20° C. then it was warmed up to 20° C., a 10% hydrochloric acid solution (560 mL) was added and the aqueous layer was extracted with AcOEt (2×750 mL).

The combined organic extracts were washed with 5% sodium hydrogen carbonate solution (600 mL) and brine (600 mL) then partially concentrated in vacuo. CH (400 mL) was added drop-wise over 1 hour at 20° C. and the resulting mixture was stirred 30 minutes and then filtered to give the title compound as a white solid (66 g).

IR (nujol, cm⁻¹): 1726 and 1655 (C═O), 1608 (C═C).

NMR (d₆-DMSO): δ (ppm) 8.19 (d, 1H); 7.31-7.18 (m, 5H); 7.08 (m, 2H); 6.94 (dt, 1H); 5.77 (d, 1H); 5.36 (d, 1H); 5.16 (2d, 2H); 3.26 (dd, 1H); 2.32 (d, 1H); 2.26 (s, 3H).

MS (ES/+): m/z=340 [MH]⁺.

### Intermediate 7

### 2-(4-Fluoro-2-methyl-phenyl)-piperidine-4-one

### Method A

2-Methyl-4-fluoro-benzaldehyde (4 g) was added to a solution of 4-aminobutan-2-one ethylene acetal (3.8 g) in dry benzene (50 mL) and the solution was stirred at r.t. under a nitrogen atmosphere. After 1 hour the mixture was heated at reflux for 16 hours and then allowed to cool to r.t. This solution was slowly added to a refluxing solution of p-toluensuiphonic acid (10.6 g) in dry benzene (50 mL) previously refluxed for 1 hour with a Dean-Stark apparatus. After 3.5 hours the crude solution was cooled and made basic with a saturated potassium carbonate solution and taken up with AcOEt (50 mL). The aqueous phase was extracted with AcOEt (3×50 mL) and Et2O (2×50 mL). The organic layer was dried and concentrated in vacuo to a yellow thick oil as residue (7.23 g). A portion of the crude mixture (3 g) was dissolved in a 6N hydrochloric acid solution (20 mL) and stirred at 60° C. for 16 hours. The solution was basified with solid potassium carbonate and extracted with DCM (5×50 mL). The combined organic phases were washed with brine (50 mL), dried and concentrated in vacuo to give the title compound (2.5 g) as a thick yellow oil.

### Method B

L-selectride (1 M solution in dry THF, 210 mL) was added drop-wise, over 80 minutes, to a solution of intermediate 6 (50 g) in dry THF (1065 mL) previously cooled to −72° C. under a nitrogen atmosphere. After 45 minutes, 2% sodium hydrogen carbonate solution (994 mL) was added drop-wise and the solution was extracted with AcOEt (3×994 mL). The combined organic phases were washed with water (284 mL) and brine (568 mL). The organic phase was dried and concentrated in vacuo to get 1-benzyloxycarbonyl-2-(4-fluoro-2-methyl-phenyl)-piperidine-4-one as a pale yellow thick oil (94 g) which was used as a crude.

This material (94 g) was dissolved in AcOEt (710 mL), then 10% Pd/C (30.5 g) was added under a nitrogen atmosphere. The slurry was hydrogenated at 1 atmosphere for 30 minutes. The mixture was filtered through Celite and the organic phase was concentrated in vacuo to give the crude 2-(4-fluoro-2-methyl-phenyl)-piperidine-4-one as a yellow oil. This material was dissolved in AcOEt (518 mL) at r.t. and racemic camphorsulphonic acid (48.3 g) was added. The mixture was stirred at r.t for 18 hours, then the solid was filtered off, washed with AcOEt (2×50 mL) and dried in vacuo for 18 hours to give 2-(4-fluoro-2-methyl-phenyl)-piperidine-4-one, 10-camphorsulfonic acid salt as a pale yellow solid (68.5 g). (M.p.: 167-169° C.—NMR (d₆-DMSO): δ (ppm) 9.43 (bs, 1H); 9.23 (bs, 1H); 7.66 (dd, 1H); 7.19 (m, 2H); 4.97 (bd, 1H); 3.6 (m, 2H); 2.87 (m, 3H); 2.66 (m, 1H); 2.53 (m, 2H); 2.37 (s+d, 4H); 2.22 (m, 1H); 1.93 (t, 1H); 1.8 (m, 2H); 1.26 (m, 2H); 1.03 (s, 3H); 0.73 (s, 3H).

This material (68.5 g) was suspended in AcOEt (480 mL) and stirred with a saturated sodium hydrogen carbonate (274 mL). The organic layer was separated and washed with further water (274 mL). The organic phase was dried and concentrated in vacuo to give the title compound (31 g) as a yellow-orange oil.

NMR (d₆-DMSO): δ (ppm) 7.49 (dd, 1H); 7.00 (m, 2H); 3.97 (dd, 1H); 3.27 (m, 1H); 2.82 (dt, 1H); 2.72 (bm, 1H); 2.47 (m, 1H); 2.40 (m, 1H); 2.29 (s, 3H); 2.25 (dt, 1H); 2.18 (m, 1H).

MS (ES/+): m/z=208 [MH]⁺.

### Intermediate 8

### 2-(R)-(4-Fluoro-2-methyl-phenyl)-piperidin-4-one mandelic acid.

A solution of L-(+)-mandelic acid (22.6 g) in AcOEt (308 mL) was added to a solution of intermediate 7 (31 g) in AcOEt (308 mL). Then isopropanol (616 mL) was added and the solution was concentrated in vacuo to 274 mL. The solution was then cooled to 0° C. and further cold isopropanol (96 mL) was added. The thick precipitate was stirred under nitrogen for 5 hours at 0° C., then filtered and washed with cold Et2O (250 mL) to give the title compound as a pale yellow solid (20.3 g).

M.p.: 82-85° C.

NMR (d₆-DMSO): δ (ppm) 7.51 (dd, 1H); 7.40 (m, 2H); 7.32 (m, 2H); 7.26 (m, 1H); 7.0 (m, 2H); 4.95 (s, 1H); 4.04 (dd, 1H); 3.31 (m, 1H); 2.88 (m, 1H); 2.49-2.2 (m, 4H); 2.29 (s, 3H).

Chiral HPLC: HP 1100 HPLC system; column Chiralcel OD-H, 25 cm×4.6 mm; mobile phase: n-hexane/isopropanol 95:5+1% diethylamine; flow: 1.3 ml/min; detection: 240/215 nm; retention time 12.07 minutes.

### Intermediate 9

### 2-(R)-(4-Fluoro-2-methyl-phenyl)-4-oxo-piperidine-1-carboxylic acid, [1-(R)-3,5-bis-trifluoromethyl-phenyl)-ethyl]-methylamide (9a) and

### 2-(S)-(4-Fluoro-2-methyl-phenyl)-4-oxo-piperidine-1-carboxylic acid, [1-(R)-3,5-bis-trifluoromethyl-phenyl)-ethyl]-methylamide (9b)

### Method A

A solution of triphosgene (147 mg) dissolved in dry DCM (5 mL) was added drop-wise to a solution of intermediate 7 (250 mg) and DIPEA (860 μL) in dry DCM (15 mL) previously cooled to 0° C. under a nitrogen atmosphere. After 2 hours, [1-(R)-3,5-bis-trifluoromethyl-phenyl)-ethyl]-methylamine hydrochloride (503 mg) and DIPEA (320 μL) in dry acetonitrile (20 mL) were added and the mixture was heated to 70° C. for 16 hours. Further [1-(R)-(3,5-bis-trifluoromethyl-phenyl)-ethyl]-methylamine hydrochloride (170 mg) and DIPEA (100 μL) were added and the mixture was stirred at 70° C. for further 4 hours. Next, the mixture was allowed to cool to r.t., taken up with AcOEt (30 mL), washed with a 1N hydrochloric acid cold solution (3×15 mL) and brine (2×10 mL). The organic layer was dried and concentrated in vacuo to a residue, which was purified by flash chromatography (CH/AcOEt 8:2) to give:
1. intermediate 9a (230 mg) as a white foam,
2. intermediate 9b (231 mg) as a white foam.

### Intermediate 9a

NMR (d₆-DMSO): δ (ppm) 7.98 (bs, 1H); 7.77 (bs, 2H); 7.24 (dd, 1H); 6.97 (dd, 1H); 6.89 (m, 1H); 5.24 (t, 1H); 5.14 (q, 1H); 3.61 (m, 1H); 3.55 (m, 1H); 2.71 (m, 2H); 2.56 (s, 3H); 2.50 (m, 2H); 2.26 (s, 3H); 1.57 (d, 3H).

### Intermediate 9b

NMR (d₆-DMSO): δ (ppm) 7.96 (bs, 1H); 7.75 (bs, 2H); 7.24 (dd, 1H); 6.98 (dd, 1H); 6.93 (dt, 1H); 5.29 (q, 1H); 5.24 (t, 1H); 3.56 (m, 1H); 3.48 (m, 1 H); 2.70 (s, 3H); 2.50 (m, 4H); 2.26 (s, 3H); 1.54 (d, 3H).

### Intermediate 9a

### Method B

A saturated sodium hydrogen carbonate solution (324 mL) was added to a solution of intermediate 8 (21.6 g) in AcOEt (324 mL) and the resulting mixture was vigorously stirred for 15 minutes. The aqueous layer was back-extracted with further AcOEt (216 mL) and the combined organic extracts were dried and concentrated in vacuo to give 2-(R)-(4-fluoro-2-methyl-phenyl)-piperidin-4-one as a yellow oil, which was treated with TEA (19 mL) and AcOEt (114 mL). The solution obtained was added drop-wise over 40 minutes to a solution of triphosgene (8 g) in AcOEt (64 mL) previously cooled to 0° C. under a nitrogen atmosphere, maintaining the temperature between 0° C. and 8° C.

After stirring for 1 hours at 0° C. and for 3 hours at 20° C., [1-(R)-(3,5-bis-trifluoromethyl-phenyl)-ethyl]-methylamine hydrochloride (29.7 g), AcOEt (190 mL) and TEA (38 mL) were added to the reaction mixture which was then heated to reflux for 16 hours.

The solution was washed with 10% sodium hydroxide solution (180 mL), 1% hydrochloric acid solution (4×150 mL), water (3×180 mL) and brine (180 mL). The organic layer was dried and concentrated in vacuo to a residue, which was purified through a silica pad (CH/AcOEt 9:1) to give the title compound (21.5 g) as a brown thick oil.

NMR (d₆-DMSO): δ (ppm) 7.97-7.77 (bs+bs, 3H); 7.24 (dd, 1H); 6.97 (dd, 1H); 6.88 (td, 1H); 5.24 (m, 1H); 5.14 (q, 1H); 3.58 (m, 2H); 2.7 (m, 2H); 2.56 (s, 3H); 2.49 (m, 2H); 2.26

### REPRESENTATIVE EXAMPLE 2

### 4-(S)-(4-Acetyl-piperazin-1-yl)-2-(R)-(4-fluoro-2-methyl-phenyl)-piperidine-1-carboxylic acid, [1-(R)-(3,5-bis-trifluoromethyl-phenyl)-ethyl]-methylamide methanesulphonate

A solution of intermediate 9a (7.7 g) in acetonitrile (177 mL) was added to a solution of 1-acetyl-piperazine (3.9 g) in acetonitrile (17.7 mL) followed by sodium triacetoxyborohydride (6.4 g) under a nitrogen atmosphere.

The reaction mixture was stirred at room temperature for 24 hours and then quenched with a saturated sodium hydrogen carbonate (23.1 mL) and water (61.6 mL). The resulting solution was concentrated in vacuo, then AcOEt (208 mL) was added; the layers were separated and the aqueous layer was back-extracted with further AcOEt (2×77 mL). The collected organic phases were washed with brine (2×118 mL), dried and concentrated in vacuo to give the crude mixture of syn and anti diastereomers (nearly 1:1) as a white foam (9.5 g).

A solution of this intermediate in THF (85.4 mL) was added to a solution of methansulfonic acid (0.890 mL) in THF (6.1 mL) at r.t.. After seeding, the desired syn diastereomer started to precipitate. The resulting suspension was stirred for 3 hours at 0° C. and then filtered under a nitrogen atmosphere. The resulting cake was washed with cold THF (15.4 mL) and dried in vacuo at +20° C. for 48 hours to give the title compound as a white solid (4.44 g).

NMR (d₆-DMSO): δ (ppm) 9.52 (bs, 1H); 7.99 (bs, 1H); 7.68 (bs, 2H); 7.23 (m, 1H); 6.95 (dd, 1H); 6.82 (m, 1H); 5.31 (q, 1H); 4.45 (bd, 1H); 4.20 (dd, 1H); 3.99 (bd, 1H); 3.65-3.25 (bm, 5H); 3.17 (m, 1H); 2.96 (m, 1H); 2.88-2.79 (m+m, 2H); 2.73 (s, 3H); 2.36 (s, 3H); 2.30 (s, 3H); 2.13-2.09 (bd+bd, 2H); 2.01 (s, 3H); 1.89-1.73 (m+m, 2H); 1.46 (d, 3H).

m.p 243.0° C.

The compound is isolated in a crystalline form.

### Intermediate 10

### 1-(Benzyloxycarbonyl)-2-(4-fluoro-2-methyl-phenyl)-2,3-dihydro-4-pyridone

A small amount of iodine was added to a suspension of magnesium turnings (13.2 g) in dry THF (300 mL), at r.t., under a nitrogen atmosphere, then the mixture was vigorously refluxed for 20 minutes. To this suspension, a 15% of a solution of 2-bromo-5-fluoro-toluene (52.5 mL) in anhydrous THF (300 mL) was added. The suspension was heated under vigorous reflux until the brown colour disappeared. The remaining part of the bromide solution was added drop-wise over 1 hour to the refluxing suspension which was then stirred for a further 1 hour. This solution of Grignard reagent was then added dropwise to the pyridinium salt obtained from benzyl chloroformate (48.7 mL) and 4-methoxypyridine (25 mL) in dry THF (900 mL) at −23° C.

The obtained solution was stirred 1 hour at −20° C. then it was warmed up to 20° C., a 10% hydrochloric acid solution (560 mL) was added and the aqueous layer was extracted with AcOEt (2×750 mL).

The combined organic extracts were washed with 5% sodium hydrogen carbonate solution (600 mL) and brine (600 mL) then partially concentrated in vacuo.

CH (400 mL) was added drop-wise over 1 hour at 20° C. and the resulting mixture was stirred 30 minutes and then filtered to give the title compound as a white solid (66 g).

IR (nujol, cm⁻¹): 1726 and 1655 (C═O), 1608 (C═C).

NMR (d₆-DMSO): δ (ppm) 8.19 (d, 1H); 7.31-7.18 (m, 5H); 7.08 (m, 2H); 6.94 (dt, 1H); 5.77 (d, 1H); 5.36 (d, 1H); 5.16 (2d, 2H); 3.26 (dd, 1H); 2.32 (d, 1H); 2.26 (s, 3H).

MS (ES/+): m/z=340 [MH]⁺.

### Intermediate 11

### 2-(4-Fluoro-2-methyl-phenyl)-piperidine-4-one

### Method A:

4-Fluoro-2-methyl-benzaldehyde (4 g) was added to a solution of 4-aminobutan-2-one ethylene acetal (3.8 g) in dry benzene (50 mL) and the solution was stirred at r.t. under a nitrogen atmosphere. After 1 hour the mixture was heated at reflux for 16 hours and then allowed to cool to r.t. This solution was slowly added to a refluxing solution of p-toluenesulphonic acid (10.6 g) in dry benzene (50 mL) previously refluxed for 1 hour with a Dean-Stark apparatus. After 3.5 hours the crude solution was cooled and made basic with a saturated potassium carbonate solution and taken up with AcOEt (50 mL). The aqueous phase was extracted with AcOEt (3×50 mL) and Et2O (2×50 mL). The organic layer was dried and concentrated in vacuo to a yellow thick oil as residue (7.23 g). A portion of the crude mixture (3 g) was dissolved in a 6N hydrochloric acid solution (20 mL) and stirred at 60° C. for 16 hours. The solution was basified with solid potassium carbonate and extracted with DCM (5×50 mL). The combined organic phases were washed with brine (50 mL), dried and concentrated in vacuo to give the title compound (2.5 g) as a thick yellow oil.

### Method B

L-selectride (1 M solution in dry THF, 210 mL) was added drop-wise, over 80 minutes, to a solution of intermediate 10 (50 g) in dry THF (1065 mL) previously cooled to −72° C. under a nitrogen atmosphere. After 45 minutes, 2% sodium hydrogen carbonate solution (994 mL) was added drop-wise and the solution was extracted with AcOEt (3×994 mL). The combined organic phases were washed with water (284 mL) and brine (568 mL). The organic phase was dried and concentrated in vacuo to get 1-benzyloxycarbonyl-2-(4-fluoro-2-methyl-phenyl)-piperidine-4-one as a pale yellow thick oil (94 g) which was used as a crude.

This material (94 g) was dissolved in AcOEt (710 mL), then 10% Pd/C (30.5 g) was added under a nitrogen atmosphere. The slurry was hydrogenated at 1 atmosphere for 30 minutes. The mixture was filtered through Celite and the organic phase was concentrated in vacuo to give the crude 2-(4-fluoro-2-methyl-phenyl)-piperidine-4-one as a yellow oil. This material was dissolved in AcOEt (518 mL) at r.t. and racemic camphorsulphonic acid (48.3 g) was added. The mixture was stirred at r.t for 18 hours, then the solid was filtered off, washed with AcOEt (2×50 mL) and dried in vacuo for 18 hours to give 2-(4-fluoro-2-methyl-phenyl)-piperidine-4-one, 10-camphorsulfonic acid salt as a pale yellow solid (68.5 g). (M.p.: 167-169° C.—NMR (d₆-DMSO): δ (ppm) 9.43 (bs, 1H); 9.23 (bs, 1H); 7.66 (dd, 1H); 7.19 (m, 2H); 4.97 (bd, 1H); 3.6 (m, 2H); 2.87 (m, 3H); 2.66 (m, 1H); 2.53 (m, 2H); 2.37 (s+d, 4H); 2.22 (m, 1H); 1.93 (t, 1H); 1.8 (m, 2H); 1.26 (m, 2H); 1.03 (s, 3H); 0.73 (s, 3H).

This material (68.5 g) was suspended in AcOEt (480 mL) and stirred with a saturated sodium hydrogen carbonate (274 mL). The organic layer was separated and washed with further water (274 mL). The organic phase was dried and concentrated in vacuo to give the title compound (31 g) as a yellow-orange oil.

NMR (d₆-DMSO): δ (ppm) 7.49 (dd, 1H); 7.00 (m, 2H); 3.97 (dd, 1H); 3.27 (m, 1H); 2.82 (dt, 1H); 2.72 (bm, 1H); 2.47 (m, 1H); 2.40 (m, 1H); 2.29 (s, 3H); 2.25 (dt, 1H); 2.18 (m, 1H).

MS (ES/+): m/z=208 [MH]⁺.

### Intermediate 12

### 2-(R)-(4-Fluoro-2-methyl-phenyl)-4-oxo-piperidine-1-carboxylic acid [1 -(R)-3,5-bis-trifluoromethyl-phenyl)-ethyl]-methylamide (12a)

and

### 2-(S)-(4-Fluoro-2-methyl-phenyl)-4-oxo-piperidine-1-carboxylic acid [1 -(R)-3,5-bis-trifluoromethyl-phenyl)-ethyl]-methylamide (12b).

### Method A:

A solution of triphosgene (147 mg) dissolved in dry DCM (5 mL) was added drop-wise to a solution of intermediate 11 (250 mg) and DIPEA (860 μL) in dry DCM (15 mL) previously cooled to 0° C. under a nitrogen atmosphere. After 2 hours, [1-(R)-3,5-bis-trifluoromethyl-phenyl)-ethyl]-methylamine hydrochloride (503 mg) and DIPEA (320 μL) in dry acetonitrile (20 mL) were added and the mixture was heated to 70° C. for 16 hours. Further [1-(R)-(3,5-bis-trifluoromethyl-phenyl)ethyl]-methylamine hydrochloride (170 mg) and DIPEA (100 μL) were added and the mixture was stirred at 70° C. for further 4 hours. Next, the mixture was allowed to cool to r.t., taken up with AcOEt (30 mL), washed with a 1N hydrochloric acid cold solution (3×15 mL) and brine (2×10 mL). The organic layer was dried and concentrated in vacuo to a residue, which was purified by flash chromatography (CH/AcOEt 8:2) to give:
3. intermediate 12a (230 mg) as a white foam,
4. intermediate 12b (231 mg) as a white foam.

### Intermediate 12a

NMR (d₆-DMSO): δ (ppm) 7.98 (bs, 1H); 7.77 (bs, 2H); 7.24 (dd, 1H); 6.97 (dd, 1H); 6.89 (m, 1H); 5.24 (t, 1H); 5.14 (q, 1H); 3.61 (m, 1H); 3.55 (m, 1H); 2.71 (m, 2H); 2.56 (s, 3H); 2.50 (m, 2H); 2.26 (s, 3H); 1.57 (d, 3H).

### Intermediate 12b

NMR (d₆-DMSO): δ (ppm) 7.96 (bs, 1H); 7.75 (bs, 2H); 7.24 (dd, 1H); 6.98 (dd, 1H); 6.93 (dt, 1H); 5.29 (q, 1H); 5.24 (t, 1H); 3.56 (m, 1H); 3.48 (m, 1H); 2.70 (s, 3H); 2.50 (m, 4H); 2.26 (s, 3H); 1.54 (d, 3H).

### Intermediate 12a

### Method B

A saturated sodium hydrogen carbonate solution (324 mL) was added to a solution of intermediate 13 (21.6 g) in AcOEt (324 mL) and the resulting mixture was vigorously stirred for 15 minutes. The aqueous layer was back-extracted with further AcOEt (216 mL) and the combined organic extracts were dried and concentrated in vacuo to give 2-(R)-(4-fluoro-2-methyl-phenyl)-piperidin-4-one as a yellow oil, which was treated with TEA (19 mL) and AcOEt (114 mL). The solution obtained was added drop-wise over 40 minutes to a solution of triphosgene (8 g) in AcOEt (64 mL) previously cooled to 0° C. under a nitrogen atmosphere, maintaining the temperature between 0° C. and 8° C. After stirring for 1 hours at 0° C. and for 3 hours at 20° C., [1-(R)-(3,5-bis-trifluoromethylphenyl)-ethyl]-methylamine hydrochloride (29.7 g), AcOEt (190 mL) and TEA (38 mL)were added to the reaction mixture which was then heated to reflux for 16 hours.

The solution was washed with 10% sodium hydroxide solution (180 mL), 1% hydrochloric acid solution (4×150 mL), water (3×180 mL) and brine (180 mL). The organic layer was dried and concentrated in vacuo to a residue, which was purified through a silica pad (CH/AcOEt 9:1) to give the title compound (21.5 g) as a brown thick oil.

NMR (d₆-DMSO): δ (ppm) 7.97-7.77 (bs+bs, 3H); 7.24 (dd, 1H); 6.97 (dd, 1H); 6.88 (td, 1H); 5.24 (m, 1H); 5.14 (q, 1H); 3.58 (m, 2H); 2.7 (m, 2H); 2.56 (s, 3H); 2.49 (m, 2H); 2.26

### Intermediate 13

### 2-(R)-(4-Fluoro-2-methyl-phenyl)-piperidin-4-one L-(+)-mandelate

A solution of L-(+)-mandelic acid (22.6 g) in AcOEt (308 mL) was added to a solution of intermediate 11 (31 g) in AcOEt (308 mL). Then isopropanol (616 mL) was added and the solution was concentrated in vacuo to 274 mL. The solution was then cooled to 0° C. and further cold isopropanol (96 mL) was added. The thick precipitate was stirred under nitrogen for 5 hours at 0° C., then filtered and washed with cold Et20 (250 mL) to give the title compound as a pale yellow solid (20.3 g).

M.p.: 82-85° C.

NMR (d₆-DMSO): δ (ppm) 7.51 (dd, 1H); 7.40 (m, 2H); 7.32 (m, 2H); 7.26 (m, 1H); 7.0 (m, 2H); 4,95 (s, 1H); 4.04 (dd, 1H); 3.31 (m, 1H); 2.88 (m, 1H); 2.49-2.2 (m, 4H); 2.29 (s, 3H).

Chiral HPLC: HP 1100 HPLC system; column Chiralcel OD-H, 25 cm×4.6 mm; mobile phase: n-hexane/isopropanol 95:5+1% diethylamine; flow: 1.3 ml/min; detection: 240/215nm; retention time 12.07 minutes.

### Intermediate 14

### 1,4-Dibenzyl-2-piperazinecarboxaldehyde

A solution of ethyl 2,3-dibromopropionate (6 mL) in anhydrous toluene (50 mL) was added to a solution of N,N′-dibenzylethylenediamine (5 g) and DIPEA (12 mL) in anhydrous toluene (50 mL) under a Nitrogen atmosphere. The resulting mixture was heated to 100° C. for 21 hours, then allowed to cool to r.t., diluted with AcOEt (100 mL) and washed with brine (3×100 mL). The organic extract was dried and concentrated in vacuo to a residue which was purified by flash chromatography (CH/AcOEt 9:1) to give ethyl 1,4-dibenzyl-piperazine-2-carboxylate (5.65 g) as a yellow oil, which was used without any purification in the next step.

Diisobutylaluminium hydride (1 M in toluene—29 mL) was dropped into a solution of ethyl 1,4-dibenzyl-piperazine-2-carboxylate (5.47 g) in anhydrous toluene (110 mL) previously cooled to −78° C. under a Nitrogen atmosphere. The solution was stirred at −78° C. for 1 hour, then a 20% sodium hydroxide solution (20 mL) was added and the mixture was allowed to warm to r.t.. Further 20% sodium hydroxide solution (50 mL) was added and the solution was extracted with Et2O (2×150 mL). The combined organic extracts were dried and concentrated in vacuo to give the title compound (5.33 g) as a crude, which was used without any further purification in the next step.

T.I.c.: CH/AcOEt 8:2, Rf=0.36.

NMR (d₆-DMSO): δ (ppm) 9.62 (s, 1H); 7.4-7.15 (m, 10H); 3.86 (d, 1H); 3.6 (d, 1H); 3.46 (s, 2H); 3.09 (bt, 1 H); 2.82 (t, 1 H); 2.55-2.45 (m, 2H); 2.4-2.3 (m, 3H).

### Intermediate 15

### Hexahydro-pyrrolo[1,2-a]pyrazin-6-one

### Method A:

(Carbethoxymethylene)triphenylphosphorane (11.72 g) was added in two portions to a solution of intermediate 14 (4.95 g) in anhydrous toluene (100 mL) under a Nitrogen atmosphere. The mixture was heated to 80° C. for 24 hours, then it was allowed to cool to r.t. and washed with water (100 mL). The organic layer was dried and concentrated in vacuo to a residue, which was purified by flash chromatography (CH/AcOEt 85:15) to give ethyl 1,4-dibenzyl-2-piperazine-3-acrylate (4.2 g—T.I.c.: CH/AcOEt 8:2, Rf=0.36).

A solution of ethyl 1,4-dibenzyl-2-piperazine-3-acrylate (2.84 g) in abs. EtOH (40 mL) was hydrogenated over Pd/C 10% (1.42 g) at 3.5 atm. for 2 days. After filtration, the solution was concentrated to nearly 30 mL and heated to 70° C. for 16 hours until complete cyclization occurred. The solution was concentrated in vacuo and the residue was purified by flash chromatography (DCM/MeOH 7:3) to give the title compound (820 mg) as a pale yellow oil.

### Method B:

Diisobutylaluminium hydride (1.2M in toluene—262 mL) was dropped into a solution of ethyl 1,4-dibenzyl-piperazine-2-carboxylate (48.4 g) synthesised as previously described in anhydrous toluene (450 mL) previously cooled to −78° C. under a Nitrogen atmosphere (addition of DIBAL-H took 1.5 hours and the internal temperature was always maintained below −70° C.). The solution was stirred at −78° C. for 2 hour, then a 10% sodium hydroxide solution (500 mL) was added and the mixture was allowed to warm to r.t.. Further 10% sodium hydroxide solution (400 mL) was added and the solution was extracted with toluene (2×250 mL). The combined organic extracts were dried and concentrated in vacuo until a volume of ˜100 mL containing 1,4-dibenzyl-2-piperazinecarboxaldehyde, which was used without any further purification in the next step.

(Carbethoxymethylene)triphenylphosphorane (75 g) was added in two portions to the previous solution of 1,4-dibenzyl-2-piperazine carboxaldehyde in toluene (450 mL) under a Nitrogen atmosphere. The mixture was heated to 80° C. overnight, then it was allowed to cool to r.t. and washed with water (2×400 mL) and brine (250 mL) The organic layer was dried and concentrated in vacuo to a residue, which was purified by flash chromatography (CH/AcOEt 85:15) to give ethyl 1,4-dibenzyl-2-piperazine-3-acrylate (44.8 g—T.I.c.: CH/AcOEt 8:2, Rf=0.36).

To a solution of ethyl 1,4-dibenzyl-2-piperazine-3-acrylate (44.8 g) in MeOH (450 mL) under a Nitrogen atmosphere, ammonium formate (23.2 g) and 5% palladium on charcoal (8.96 g) were added. The resulting mixture was heated to reflux temperature for 6 h. After filtration over Celite, the solution was concentrated in vacuo and the residue was purified by flash chromatography (DCM/MeOH 8:2) to give the title compound (14.15 g) as a pale yellow oil.

### Method C:

Intermediate 17 (820 g) and toluene (1680 g) were charged in a 5 L stainless steel autoclave and palladium on charcoal (5%, dry—50 g) was added. The autoclave was rendered inert with nitrogen, subsequently filled with 100 bar hydrogen, and then heated to 100° C. When the internal pressure has fallen to 90 bar, the pressure was increased to 100 bar again. After the hydrogen uptake ceased, the autoclave was cooled below 30° C. and the reaction solution was removed. The catalyst was then filtered off with a buchner funnel and washed with toluene (2×200 mL). After concentrating the filtrate with a rotatory evaporator, the product was distilled over a 15 cm Vigreux column (bp: 115 to 125 ° C. @ 0.07 mbar) giving the title compound (574 g) as a slightly yellowish oil.

T.I.c.: DCM/MeOH 7:3, Rf=0.17 (detection with ninhydrin)

NMR (CDCl3): δ (ppm) 4.01(m, 1H); 3.54 (m, 1H); 3.16 (m, 1H); 3.01 (m, 1H); 2.81 (m, 1H); 2.6 (dt, 1H); 2.38 (m, 3H); 2.16 (m, 1H); 1.6 (m, 1H).

MS (ES/+): m/z=141 [M+H]⁺

### Intermediates 16

### (8aS)-Hexahydro-pyrrolo[1,2-a]pyrazin-6-one (16a)

and

### (8aR)-Hexahydro-pwrolo[1,2-a]pyrazin-6-one (16b)

### Method A:

Intermediate 15 (746 mg) was separated into the enantiomers via preparative HPLC (Column: Chiralpack AD 25×2 cm; mobile phase: n-hexane/EtOH 8:2; flux=1 mL/min; λ=225 nm). Thus, intermediate 16a (330 mg) and intermediate 16b (320 mg) were obtained.

### Intermediate 16a (Enantiomer 1):

HPLC: Column Chiralpack AD 25cm×4.6mm×5μ; mobile phase n-hexane/EtOH 8:2; flux=1 mL/min; λ=225 nm; retention time 10.7 minutes. Ratio 16a/16b=100:0.

### Intermediate 16b (Enantiomer 2):

HPLC: Column Chiralpack AD 25cm×4.6mm×5μ; mobile phase n-hexane/EtOH 8:2; flux=1 mL/min; λ=225 nm; retention time 12.8 minutes. Ratio 16a/16b=0:100.

### Intermediate 16b:

### Method B:

A solution of L-(+)-mandelic acid (13.03 g) in isopropanol (60 mL) was dropped over 20 minutes into a solution of intermediate 15 (12 g) in isopropanol (60 mL) under a Nitrogen atmosphere. The suspension was stirred at 23° C. for 2 hours, then it was filtered off and washed with further isopropanol (120 mL). The solid obtained (ratio enantiomers 20:80) was recrystallized three times from isopropanol (10 volumes) until a complete HPLC enantioselectivity was detected. In this way, (8aR)-hexahydro-pyrrolo[1,2-a]pyrazin-6-one L-(+)-mandelate (5.84 g—enantiomer 2) was obtained.

This material (6.469 g) was dissolved in EtOH (40 mL) and water (4 mL) and stirred with a suspension of resin IRA68 (112 g—previously washed with a 0.05N sodium hydroxide solution (370 mL) and water (4 L) until neutral pH) in EtOH (200 mL). The mixture was stirred at 23° C. for 1.5 hours, then filtered off. The organic layer was concentrated in vacuo to give the title compound (3.1 g) as a white solid.

### Intermediate 16b:

HPLC: Column Chiralpack AD 25cm×4.6mm×5μ; mobile phase n-hexane/EtOH 8:2; flux=1 mL/min; λ=225 nm; retention time 12.8 minutes. Ratio 16a/16b=0:100.

### Intermediate 16a:

### Method B:

A part of the mother liquors (3.48 g with ratio 16a:16b=63:37) was treated with a suspension of resin IRA68 (70 g—previously washed with a 0.05N sodium hydroxide solution (150 mL) and water until neutral pH) in EtOH (150 mL) and water (1 mL). The mixture was stirred at 23° C. for 2 hours, then filtered off. The organic layer was concentrated in vacuo to give the free hexahydro-pyrrolo[1,2-a]pyrazin-6-one (1.6 g) as colourless oil. This material (1.6 g) was dissolved in isopropanol (8 mL) and treated with a solution of D-(−)-mandelic acid (1.74 g) in isopropanol (8 mL).

The suspension was stirred at 23° C. for 16 hours, then it was filtered off and washed with further isopropanol (120 mL). The solid obtained (ratio enantiomers 86:14) was recrystallized three times from isopropanol (10 volumes) until a complete HPLC enantioselectivity was detected. In this way, (8aS)-hexahydro-pyrrolo[1,2-a]pyrazin-6-one D-(−)-mandelate (0.88 g—enantiomer 1) was obtained.

This material (0.88 g) was dissolved in EtOH (10 mL) and water (1 mL) and stirred with a suspension of resin IRA68 (15 g—previously washed with a 0.05N sodium hydroxide solution (50 mL) and water until neutral pH in EtOH (30 mL). The mixture was stirred at 23° C. for 1 hour, then filtered off. The organic layer was concentrated in vacuo to give the title compound (0.434 g) as a white solid.

### Intermediate 16a:

HPLC: Column Chiralpack AD 25cm×4.6mm×5μ; mobile phase n-hexane/EtOH 8:2; flux=1 mL/min; λ=225 nm; retention time 10.7 minutes. Ratio 16a/16b=100:0.

### Intermediate 17

### 3-Pyrazin-2-yl-propionic acid ethyl ester

Butyl lithium (2.5M in hexane—2560 mL) was added within 2 hours into a 10 L flask charged with THF (3350 mL) and diisopropylamine (658 g) while the temperature was maintained at 0-5° C. with an ice bath. The LDA solution was then precooled to −50° C. and a mixture of methylpyrazine (606 g) and THF (590 mL) was added within 2 hours under vigorous stirring at −40 to −30° C. The deep red anion solution is then pumped to a cooled (−60° C.) mixture of tert-butyl bromoacetate (1255 g) and THF (3360 mL) in a 20 L reactor. During the addition of the anion solution, the temperature in the reaction vessel did not exceed −55° C. After the addition, the mixture is stirred for further 30 min at −55° C. and then transferred to a 30 L reactor (the transesterification and removal of solvents can be done for two runs at once). A solution of sodium ethylate (142 g) dissolved in EtOH (2200 mL) was then added to the orange mixture and about 12 L of solvents were distilled off until a temperature of 80° C. was reached in the distillation head and 100° C. in the boiling liquid. The mixture was cooled to approximately 30° C. and then toluene (840 mL), AcOEt (840 mL), and water (1180 mL) were added. After separation of the phases, the organic layer was extracted three times with AcOEt (420 mL) and toluene (170 mL) each. The combined organic phases were then concentrated in vacuo and the residue was distilled over a Vigreux column (bp 115 to 130° C. @ 0.07 mbar) giving the title compound (579 g).

T.I.c.:CH/EtOAc=1:1, Rf=0.36.

¹H-NMR (d₆-DMSO): δ (ppm) 8.57 (d, 1H); 8.52 (dd, 1H); 8.45 (d, 1H); 4.01 (q, 2H); 3.04 (t, 2H); 2.76 (t, 2H); 1.12 (t, 3H).

MS (ES/+): m/z=181 [M+H]⁺

### Intermediate 18

### (8aS)-Hexahydro-pyrrolo[1,2-a]pyrazin-6-one S-(+)-O-acetylmandelate (enantiomer 1)

A solution of (S)-(+)-O-acetylmandelic acid (2.77 g) in acetone (12 mL) was added dropwise to a solution of intermediate 15 (4 g) in acetone (28 mL) at 20° C. The resulting mixture was seeded to initiate the precipitation.

The obtained precipitate was stirred at 20° C. over 4 hours then filtered washing with acetone (12 mL). The solid was dried in vacuo at 40° C. for 18 hours to give the title compound (3.44 g) as a white solid.

HPLC: Column Chiralpack AD 25×4.6×5 μm; mobile phase n-hexane/EtOH=1:1; flow=1 ml/min; λ=210 nm; retention times title compound 5.42 min., (8aR) enantiomer 6.06 min. E.e.>94%.

¹H-NMR (d₆-DMSO): δ (ppm) 9.5 (broad, 1H); 7.42 (m, 2H); 7.32 (m, 3H); 5.62 (s, 1H); 3.79 (dd, 1H); 3.55 (m, 1H); 3.14 -3.02 (2dd, 2H); 2.80 (dt, 1H); 2.52 (dt, 1H); 2.40 (t, 1H); 2.19 (m, 2H); 2.06 (s, 3H); 2.05 (m, 1H); 1.49 (m, 1H).

MS (ES/+): m/z=141 [M+H—PhCH(OAc)COOH]⁺.

### REPRESENTATIVE EXAMPLE 3

### 2-(R)-(4-Fluoro-2-methyl-phenyl)-4-(R)-((8aS)-6-oxo-hexahydro-pyrrolo[1,2-a]-pyrazin-2-yl)-piperidine-1-carboxylic acid [1-(R)-(3,5-bis-trifluoromethyl-phenyl)-ethyl]-methylamide (Example 3a) and

### 2-(R)-(4-Fluoro-2-methyl-phenyl)-4-(S)-((8aS)-6-oxo-hexahydro-pyrrolo[1,2-a]-pyrazin-2-yl)-piperidine-1-carboxylic acid [1-(R)-(3,5-bis-trifluoromethyl-phenyl)-ethyl]-methylamide (Example 3b)

### Method A:

Intermediate 12a (168 mg) and sodium triacetoxyborohydride (127 mg) were added to a solution of intermediate 16a (80 mg) in anhydrous acetonitrile (4 mL) under a Nitrogen atmosphere. The mixture was stirred at 23° C. for 14 hours. The solution was diluted with a 5% sodium hydrogen carbonate solution (5 mL) and extracted with AcOEt (2×10 mL). The combined organic extracts were dried and concentrated in vacuo to a residue which was purified by flash chromatography (AcOEt/MeOH 9:1) to give three fractions:
1. example 3a (18 mg) a a white solid
2. mixture of example 3a and 3b (160 mg)
3. example 3b (8 mg) as a white solid.

### Method B:

A solution of intermediate 16a (2.4 g) in anhydrous acetonitrile (80 mL) was added to a solution of intermediate 12a (5.7 g) in anhydrous acetonitrile (30 mL) under a Nitrogen atmosphere. Sodium triacetoxyborohydride (4.36 g) was added in three portions every 15 minutes and the mixture was stirred at 23° C. for 22 hours. The solution was diluted with water (75 mL) and a saturated sodium hydrogen carbonate solution (25 mL) and extracted with AcOEt (2×200 mL). The combined organic extracts were dried and concentrated in vacuo to a residue which was purified by flash chromatography (CH/AcOEt/MeOH 50:50:8) to give four fractions:
1. mixture example 3a and example 3b (1.27 g) in ratio 1:1
2. example 3b (1.66 g) (ratio 3a:3b=13:87)
3. example 3b (420 mg) (ratio 3a:3b=5:95)
4. example 3b (800 mg) (ratio 3a:3b=2:98)

### EXAMPLE 3a

T.I.c.:AcOEt/MeOH 8:2, Rf=0.55.

MS (ES/+) m/z=629 [M+H]⁺.

HPLC: Column Supelcosil ABZ Plus 25cm×4.6mm×5μ; mobile phase NH₄OAc 10 mmol/CH₃CN from 60:40 to 10:90 in 5 min. then NH₄OAc 10 mmol/CH₃CN for 10 min.; flux=0.8 mL/min; λ=220 nm; retention time 9.27 minutes.

### EXAMPLE 3b

T.I.c.:AcOEt/MeOH 8:2, Rf=0.48.

MS (ES/+) m/z=629 [M+H]⁺.

HPLC: Column Supelcosil ABZ Plus 25 cm×4.6 mm×5μ; mobile phase NH₄OAc 10 mmol/CH₃CN from 60:40 to 10:90 in 5 min. then NH₄OAc 10 mmol/CH₃CN for 10 min.; flux=0.8 mL/min; λ=220 nm; retention time 8.84 minutes.

### REPRESENTATIVE EXAMPLE 4

### 2-(R)-(4-Fluoro-2-methyl-phenyl)-4-(S)-((8aS)-6-oxo-hexahydro-pyrrolo[1,2-a]-pyrazin-2-yl)-piperidine-1-carboxylic acid [1-(R)-(3,5-bis-trifluoromethyl-phenyl)-ethyl]-methylamide hydrochloride

A solution of example 3b (8 mg) in dry Et2O (1 mL) was treated with hydrochloric acid (1M in Et2O—14 μL) at 0° C. under a Nitrogen atmosphere. The resulting mixture was stirred at 0° C. for 20 minutes, then the mixture was concentrated in vacuo. The precipitate was washed with pentane (2 mL) to give the title compound as a white solid (7.6 mg).

NMR (d₆-DMSO): δ (ppm) 10.22 (bs, 1H); 7.99 (s, 1H); 7.67 (s, 2H); 7.22 (dd, 1H); 6.94 (dd, 1H); 6.81 (t, 1H); 5.31 (q, 1H); 4.2 (dd, 1H); 4.0-3.86 (bm, 2H); 3.6-3.4 (m, 2H); 3.1-2.7 (m, 4H); 2.73 (s, 3H); 2.4-2.0 (m, 5H); 2.35 (s, 3H); 1.94 (m, 1H); 1.57 (d, 3H); 1.46 (d, 3H).

MS (ES/+) m/z=629 [M+H—HCl]⁺.

HPLC: Column Supelcosil ABZ Plus 25 cm×4.6 mm×5μ, mobile phase NH₄OAc 10 mmol/CH₃CN from 60:40 to 10:90 in 5 min. then NH₄OAc 10 mmol/CH₃CN from 10:90 for 10 min.; flux=0.8 mL/min; λ=220 nm; retention time 8.86 minutes.

Column X-Terra 4.6×100 mm, RP18 3.5 μm; mobile phase: eluant A: NH₄HCO₃ 5 mM (pH=8)/CH₃CN 90/10—eluant B: NH₄HCO₃ 5 mM (pH=8)/CH₃CN 10/90—Gradient: from 50% B to 100% B in 7.5 min; 100% B for 0.5 min then 50% B for 3 min.; column temp.: 40° C.; flow=1 mL/min; λ=210 nm; retention time 4.15 minutes.

### REPRESENTATIVE EXAMPLE 4a

### 2-(R)-(4-Fluoro-2-methyl-phenyl)-4-(S)-((8aS)-6-oxo-hexahydro-pyrrolo[1,2-a]-pyrazin-2-O-piperidine-1-carboxylic acid [1-(R)-(3,5-bis-trifluoromethyl-phenyl)-ethyl]-methylamide hydrochloride as anhydrous crystalline form

A 2% sodium hydroxide solution (100 mL) was added to a suspension of example 5 (10 g) in AcOEt (150 mL). Then the two phases mixture were stirred for 10 minutes and the layers were separated. The organic phase was washed with water (100 mL) and then concentrated in vacuo up to 40 mL. AcOEt (100 mL) was added to the organic phase, which was then concentrated in vacuo a second time up to 40 mL. The solution was further diluted with AcOEt (60 mL) and 5-6N hydrochloric acid in isopropanol (3 mL) was added. After 5 minutes the clear solution was seeded. Precipitation occurred in a few minutes and after further 20 minutes stirring, n-heptane (100 mL) was added in 10-15 minutes. The obtained mixture was stirred 2 hours at 20° C. The solid was then filtered, washed with AOEt/n-heptane 1/1 (60 mL) and dried in vacuo at 40° C. for 16 hours to give the title compound (8.08 g) as a white solid.

X ray powder diffraction data are reported in table 1

TABLE 1
The X-ray podwer diffraction pattern of the product of the
Example 4a in terms of d spacing is as follows

| Angle(°2 Theta) | d value(A) |
|---|---|
| 3.412 | 25.87492 |
| 6.87 | 12.85613 |
| 9.867 | 8.95664 |
| 12.877 | 6.86899 |
| 14.274 | 6.19974 |
| 15.4 | 5.74895 |
| 16.732 | 5.29424 |
| 17.323 | 5.11486 |
| 17.966 | 4.93311 |
| 18.521 | 4.78656 |
| 19.557 | 4.53525 |
| 22.12 | 4.01529 |
| 22.382 | 3.96884 |
| 24.311 | 3.65818 |
| 27.117 | 3.28566 |
| 27.836 | 3.20239 |
| 28.374 | 3.14292 |
| 28.846 | 3.0925 |
| 29.372 | 3.03835 |
| 33.9 | 2.64214 |

### REPRESENTATIVE EXAMPLE 4b

### 2-(R)-(4-Fluoro-2-methyl-phenyl)-4-(S)-((8aS)-6-oxo-hexahydro-pyrrolo[1,2-a]-pyrazin-2-yl)-piperidine-1-carboxylic acid [1-(R)-(3,5-bis-trifluoromethyl-phenyl)-ethyl]-methylamide hydrochloride as dihydrate crystalline form

To a 265 mg of example 4a, 3 ml of water was added. The suspension was stirred overnight at 25° C. and then centrifuged for 5 min at 10000 rpm. The solid was filtered using a centrifugal filter device (Millipore Ultrafree-MC 0.45 μm) to obtain the title compound (250 mg)

X ray powder diffraction data are reported in table 2

Table 2

The X-ray powder diffraction pattern of the product of the Example 4b in terms of d spacing is as follows

TABLE 2

| Angle(°2-Theta) | d value(A) |
|---|---|
| 3.233 | 27.30972 |
| 6.353 | 13.90157 |
| 12.14 | 7.28437 |
| 12.647 | 6.99378 |
| 13.282 | 6.6605 |
| 13.5 | 6.55347 |
| 15.48 | 5.71928 |
| 16.324 | 5.42557 |
| 16.779 | 5.27951 |
| 17.825 | 4.97188 |
| 19.022 | 4.66158 |
| 19.414 | 4.5685 |
| 19.901 | 4.45772 |
| 21.339 | 4.1605 |
| 21.915 | 4.05245 |
| 22.21 | 3.99923 |
| 23.161 | 3.83714 |
| 23.521 | 3.77915 |
| 24.179 | 3.67782 |
| 25.417 | 3.50136 |
| 26 | 3.42415 |
| 26.668 | 3.33994 |
| 28.052 | 3.17821 |
| 28.553 | 3.1236 |
| 29.551 | 3.0203 |
| 31.297 | 2.85568 |
| 32.8 | 2.72816 |
| 34.148 | 2.62353 |

### REPRESENTATIVE EXAMPLE 5

### 2-(R)-(4-Fluoro-2-methyl-phenyl)-4-(S)-((8aS)-6-oxo-hexahydro-pyrrolo[1,2-a]-pyrazin-2-yl)-piperidine-1 -carboxylic acid [1-(R)-(3,5-bis-trifluoromethyl-phenyl)-ethyl]-methylamide maleate

### Method A:

Intermediate 18 (25 g) was suspended in acetonitrile (300 mL), then TEA (10.4 mL) was quickly added in order to obtain the free base: the aspect of the slurry did not change as a new precipitate of TEA-acetylmandelate salt was formed. The mixture was kept under stirring for 15-20 minutes. Meanwhile intermediate 12a (25 g) was dissolved in acetonitrile (125 mL) and the so-obtained solution was quickly added to the slurry. Then Sodium triacetoxyborohydride (15 g) was added all at once and the mixture was kept under stirring conditions for 22 hours. The white precipitate was filtered off and the mother liquors were evaporated to 100 mL. AcOEt (250 mL) was added to the so-obtained mixture and the resulting solution was washed with aqueous 4% sodium hydrogen carbonate solution (2×125 mL) and then with 5% sodium chloride solution (125 mL). The organic layer dried and evaporated to 100 mL. Isopropyl alcohol (150 ml) was added and the mixture was evaporated again to 100 mL. This operation was repeated. The final volume of the mixture was adjusted to 200 mL adding further isopropyl alcohol (100 mL). A solution of maleic acid (5.8 g) in isopropyl alcohol (50 mL) was dropped in ca. 10 minutes. The mixture was seeded and precipitation occurred in few minutes. The slurry was stirred 1 hour at 20° C. and isoctane (250 mL) was added in 10 minutes. The resulting suspension was stirred at room temperature for 22 hours. The solid was filtered and washed with isopropanol/isoctane 1/1 (150 mL) and dried in vacuo at 40° C. for 18 hours giving the title compound (13.75 g) as a white solid.

### Method B:

Intermediate 18 (1 g) was suspended in acetonitrile (12 mL), then TEA (0.415 mL) was quickly added in order to obtain the free base: the aspect of the slurry did not change as a new precipitate of TEA-acetylmandelate salt was formed. After 30 minutes of stirring, the mixture was treated with sodium triacetoxyborohydride (0.6 g) plus formic acid (0.224 mL).

Meanwhile intermediate 12a (1 g) was dissolved in acetonitrile (6 mL) and the so-obtained solution was quickly added to the slurry and the resulting mixture was kept under stirring conditions for 18 hours. The slurry was evaporated to small volume. AcOEt (10 mL) was added to the so-obtained mixture and the resulting solution was washed with aqueous 4% sodium hydrogen carbonate (2×5 mL) and then with 5% sodium chloride solution (5 mL). The organic layer was dried and evaporated to a white foam.

Isopropyl alcohol (10 mL) was added and the mixture was evaporated again to dryness. The resulting foam was, once again, dissolved in isopropyl alcohol (8 mL) and treated drop-wise with a solution of maleic acid (0.232 g) in isopropyl alcohol (2 mL). After 30 minutes the mixture was seeded and precipitation occurred in a few minutes. The slurry was stirred 1 hour at 20° C. and then isoctane (10 mL) was added dropwise over 5-10 minutes. The resulting suspension was stirred at room temperature for 19 hours. The solid was filtered and washed with isopropanol/isoctane 1/1 (5 mL) and dried in vacuo at 40° C. for 18 hours giving the title compound (0.639 g) as a white solid.

HPLC: Column X-Terra 4.6×100 mm, RP18 3.5 μm; mobile phase: eluant A: NH₄HCO₃ 5 mM (pH=8)/CH₃CN 90/10—eluant B: NH₄HCO₃ 5 mM (pH=8)/CH₃CN 10/90—Gradient from 50% B to 100% B in 7.5 minutes; 100% B for 0.5 minutes then 50% B for 3 minutes; column temp. 40° C.; flow=1 mL/min; λ=210 nm; retention times 4.15 minutes, >99% a/a.

¹H-NMR (d₆-DMSO): δ (ppm) 7.98 (bs, 1H); 7.68 (bs, 2H); 7.21 (dd, 1H); 6.93 (dd, 1H); 6.81 (dt, 1H); 6.09 (s, 2H); 5.31 (q, 1H); 4.19 (dd, 1H); 3.93 (m, 1H); 3.74 (bm, 1H); 3.46 (m, 1H); 3.45 (bm, 1H); 3.30 (bm, 2H); 2.93 (bt, 1H); 2.79 (t, 1H); 2.73 (s, 3H); 2.73 (bm, 1H); 2.60 (bm, 1H); 2.35 (s, 3H); 2.23 (m, 2H); 2.12 (m, 1H); 2.04 (bd, 1H); 1.98 (bd, 1H); 1.84 (m, 1H); 1.64 (q, 1H); 1.56 (m, 1H); 1.46 (d, 3H).

MS (ES/+): m/z=629 [MH—HOOCCHCHCOOH]⁺

The ability of a compound to act as an NK1 receptor antagonist may be determined using the gerbill foot tapping model as described by Rupniak & Williams, Eur. Jour. of Pharmacol., 1994.

The compound is orally administered and four hours later an NK1 agonist (e.g. delta-Aminovaleryl⁶[Pro⁹,Me-Leu¹⁰]-substance P (7-11)) (3 pmol in 5 μL icy) is infused directly in the cerebral ventricules of the animals. The duration of hind foot tapping induced by the NK1 agonist (e.g. delta-Aminovaleryl⁶[Pro⁹,Me-Leu¹⁰]-substance P (7-11)) is recorded continuously for 3 min using a stopclock. The dose of the test compound required to inhibit by 50% the tapping induced by the NK1 agonist (e.g. delta-Aminovaleryl⁶[Pro⁹,Me-Leu¹⁰]-substance P (7-11)) expressed as mg/kg is referred to as the ED50 value. Alternatively the compounds may be administered subcutaneously or intraperitoneally.

The ability of a compound to act as an SSRI may be determined using the standard pharmacological assay as set out in Wong, et al., Neuropsychopharmacology 8, 337-344 (1993), incorporated herein by reference.

The affinity of the compounds to bind the re-uptake site of serotonin transporter (SERT) may be assessed using [3H]citalopram binding assay performed in recombinant epithelial pig kidney cells stably transfected with human SERT (hSERT/LLCPK). Grow cells in Petri dishes of 500 cm² and use for membrane preparation at 80% of confluence. Harvest cells in phosphate buffered saline (PBS) containing 5 mM EDTA and centrifuge at 900 g for 8 min at 4° C. Homogenize the pellet in 30-50 vols of assay buffer (50 mM Tris, 120 mM NaCl, 5 mM KCl, 10 μM pargyline, 0.1% ascorbate (pH=7.7)) and centrifuge at 48000 g for 20 min at 4° C. Resuspend the pellet in the same volume and after incubation at 37° C. for 20 min, centrifuge as before and finally aliquot at ˜0.2 mg protein/ml in cold assay buffer. For [3H]citalopram binding assay, add 4 μl of test compound (100 times in neat DMSO) (to define total binding) or a final concentration of 10 μM fluoxetine in DMSO (to define non-specific binding), 200 μl of [³H]citalopram at final concentration of 0.25 nM in assay buffer and 200 μl of membranes diluted in assay buffer at concentration of 2 μg/well of protein (final assay volume 400 μl). Add membranes to initiate the reaction and incubate at room temperature for 2 h. Stop the reaction by rapid filtration through GF/B 96-filterplate pre-soaked in 0.5% polyethylenimmine (PEI) using a Packard cell harvester. Wash 96-filterplate 3 times with 1 ml/well cold 0.9% NaCl solution and count the radioactivity in Packard TopCount.

### Clinical Trial A

### Study Design

A randomised, double-blind, placebo-controlled, cross-over study that investigates the effects of single dose and repeated dosing of 2-(S)-(4-fluoro-2-methyl-phenyl)-piperazine-1-carboxylic acid [1-(R)-(3,5-bis-trifluoromethyl-phenyl)-ethyl]-methyl-amide methanesulfonate-paroxetine combination and 2-(S)-(4-fluoro-2-methyl-phenyl)-piperazine-1-carboxylic acid [1-(R)-(3,5-bis-trifluoromethyl-phenyl)-ethyl]-methyl-amide methanesulfonate alone vs. placebo in an enriched population of tinnitus patients.

Subjects suffering from tinnitus will be attending the clinics for 2 screening visits, then randomized to one of the six repeated dosing treatment sequences. Treatment phase consists of 3 treatment periods of 14 days each, separated by a wash-out interval of 14 days. Assessment with self-rated scales, audiogram, and psychoacoustic tests will be performed at the Day 1 and on the 14th Day of each treatment period within 4 hrs following last dose. All subjects will receive all treatments and a follow up visit will be performed 14 days after last drug dosing. Treatments are:
1. placebo
2. 2-(S)-(4-fluoro-2-methyl-phenyl)-piperazine-1-carboxylic acid [1-(R)-(3,5-bis-trifluoromethyl-phenyl)-ethyl]-methyl-amide methanesulfonate 25 mg/day+paroxetine 20 mg/day
3. 2-(S)-(4-fluoro-2-methyl-phenyl)-piperazine-1-carboxylic acid [1-(R)-(3,5-bis-trifluoromethyl-phenyl)-ethyl]-methyl-amide methanesulfonate 25 mg/day

The main enrichment criteria of the population included is based on the presence of a mild-to-moderate auditory impairment that could be related to acoustic trauma (i.e., either as historical record, self-report, or tonal audiogram profile) and a positive response to the lidocaine test.

The first screening visit will consist of the clinical assessment for tinnitus diagnosis, including audiograms and questionnaires, and a medical screen including, ECG, laboratory tests on blood and urine, current treatment, and their agreement to participate in the study.

The second screening visit will be carried out only if the subject is found clinically eligible during the first visit. The second visit will be conducted in a quiet room where the subject will be tested for a lidocaine response. Subjects will be infused with placebo or lidocaine (1.5 mg/kg body weight) for 5 min in a randomized, balanced order, being blind of the treatment. VAS for pitch, loudness, and distress of tinnitus, and the presence of hyperacusis, will be collected at pre-infusion time and 5 min after each infusion. The two infusions will be separated by at least 20-30 min. Subjects will only be included if:
a) there is consistency between the two pre-infusion values (a difference of less than 30% between the highest and the lowest pre-infusion, baseline value)
b) there is a reduction from baseline in VAS for loudness after lidocaine of at least 10 mm greater than the reduction following placebo.

### Study Population

### Number of Subjects

Twenty-four subjects will be recruited in the study to obtain at least 19 subjects completing the study.

### Eligibility Criteria

Eligible subjects will be male or female subjects, aged between 18 and 60 years, with a confirmed diagnosis of tinnitus, based on standardized ONH visit and examination, including otoscopy and audiograms. Subjects with completely normal audiogram or those with severe hearing loss will be excluded. In cases where a disorder is associated with tinnitus, such as Meniere's Disease or Otosclerosis, subjects will also be excluded. Patients should have been suffering with tinnitus for at least six months. Enrichment criteria will be based on the documented presence of auditory impairment and the capacity to detect transient changes in tinnitus loudness to lidocaine infusion (reduction of 20% or more vs. placebo normalized to baseline).

### Endpoints

### Primary

Visual Analog Scales (VAS) to measure the change in tinnitus loudness as perceived at the moment of the measurement at 2 hrs after dosing (or at any other time point vs. pre-dose baseline).

### Secondary

1. Visual Analog Scales (VAS) to measure the level in tinnitus pitch and tinnitus distress as perceived at the moment of the measurement
2. VAS to measure arousal/anxiety (i.e., tired-energetic, active-drowsy, tense-peaceful, and worried-relaxed)
3. Pure Tone Audiometry (Audiogram)
4. Psychoacoustic assessment (automated if possible) of tinnitus pitch, timbre, intensity, Minimal Masking Level
5. Self-report questionnaires (integrated evaluation referring only to the day of testing after dosing): Tinnitus Handicap Inventory (THI)
6. Self-report questionnaires (integrated evaluation referring to the past week, up to the moment of measurement):
   a. Tinnitus Handicap Inventory (THI)
   b. Quick Inventory of Depressive Symptomatology (QIDS-SR 16).
   c. Leeds Sleep Evaluation Questionnaire (LSEQ)
7. Diary (to be filled in the evening):
   a. Number of benzodiazepines, analgesic or any other calming infuses consumed during days
   b. VAS for tinnitus loudness, pitch, and/or distress (integrated assessment of the whole day)
   c. Number of hyperacusis, brief description of the generator, and VAS distress rating of the overall distress
8. Clinician rated scales:
   a. Annoyance of Tinnitues: Question: “How aggravated are you by tinnitus? Please rate from 0 to 7 point of intensity” (8-point scale, [Robinson et al, Psychosomatic Med. 2005; 67:981-988 and Zenner et al, Acta Oto-Laryngologica. 2005: 125:1184-1188]
   b. Annoyance of Hyperacusis: Answer to questions: (i), “Do you felt hypersensitive to noise? (yes/no response); and (ii), if yes, a score >1 (on the 0-10 scale) for the overall impact of hyperacusis on everyday life [Dauman et al, Acta Oto-Laryngologica. 2005. 125:503-509].

### Clinical Trial B

### Study Design

A multi-centre, two-period cross-over, double-blind, randomized, placebo-controlled study that investigates the effects of single dose and repeated dosing of 2-(S)-(4-fluoro-2-methyl-phenyl)-piperazine-1-carboxylic acid [1-(R)-(3,5-bis-trifluoromethyl-phenyl)-ethyl]-methyl-amide methanesulfonate-paroxetine combination vs. placebo in a population of tinnitus patients.

Subjects suffering from tinnitus will be subject to two treatment periods of 14 days each, separated by a wash-out interval of 14 days. Treatments are:
1. placebo
2. 2-(S)-(4-fluoro-2-methyl-phenyl)-piperazine-1-carboxylic acid [1-(R)-(3,5-bis-trifluoromethyl-phenyl)-ethyl]-methyl-amide methanesulfonate 25 mg/day+paroxetine 20 mg/day

### Study Population

Number of Subjects

Sufficient subjects will be recruited into the study to obtain at least 35 subjects completing the study.

### Eligibility Criteria

Eligible subjects will be male or female subjects, aged between 18 and 60 years, with a confirmed diagnosis of tinnitus, based on standardized ONH visit and examination, including otoscopy and audiograms. In cases where a disorder is associated with tinnitus, such as Meniere's Disease or Otosclerosis, subjects will also be excluded. Patients should have been suffering with tinnitus for at least six months.

### Endpoints

### Primary

To measure the effect of the combination of 2-(S)-(4-fluoro-2-methyl-phenyl)-piperazine-1-carboxylic acid [1-(R)-(3,5-bis-trifluoromethyl-phenyl)-ethyl]-methyl-amide methanesulfonate 25 mg/day and paroxetine 20 mg/day vs placebo on either the change in tinnitus loudness [Visual Analog Scales (VAS)] after a single dose (on day 1) and/or after repeated dosing (on Day 14 vs Day 1 baseline), or on tinnitus matching.

1-9. (canceled)

10. A method for treating a subject suffering from tinnitus, hearing loss, or tinnitus and hearing loss comprising administering to the subject an effective amount of an NK1 receptor antagonist alone, or in combination with an effective amount of a selective serotonin reuptake inhibitor.

11. The method according to claim 10, wherein an effective amount of an NK1 receptor antagonist in combination with a selective serotonin reuptake inhibitor is administered to the subject.

12. The method according to claim 10, wherein the subject suffers from tinnitus.

13. The method according to claim 10, wherein the subject suffers from hearing loss.

14. The method according to claim 10, wherein the subject suffers from tinnitus and hearing loss.

15. The method according to claim 10, wherein the NK1 receptor antagonist comprises a compound of formula (I): 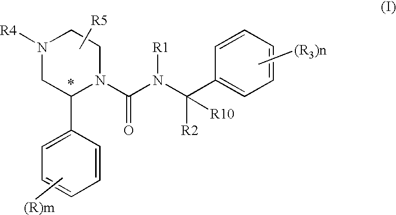
wherein,
R represents a halogen atom or a C₁₋₄ alkyl group;
R1 represents hydrogen or a C₁₋₄alkyl group;
R2 represents hydrogen, a C₁₋₄ alkyl, C₂₋₆ alkenyl or a C₃₋₇ cycloalkyl group; or R1 and R2 together with nitrogen and carbon atom to which they are attached respectively represent a 5-6 membered heterocyclic group;
R3 represents a trifluoromethyl, a C₁₋₄ alkyl, a C₁₋₄ alkoxy, a trifluoromethoxy or a halogen group;
R4 represents hydrogen, a (CH₂)_{q}R7 or a (CH₂)ᵣCO(CH₂)ₚR7 group;
R5 represents hydrogen, a C₁₋₄ alkyl or a COR6 group;
R6 represents hydrogen, hydroxy, amino, methylamino, dimethylamino a 5 membered heteroaryl group containing 1 to 3 heteroatoms selected from oxygen, sulphur and nitrogen or a 6 membered heteroaryl group containing 1 to 3 nitrogen atoms;
R7 represents hydrogen, hydroxy or NR8R9 wherein R8 and R9 represent independently hydrogen or C₁₋₄ alkyl optionally substituted by hydroxy, or by amino;
R10 represents hydrogen, a C₁₋₄ alkyl group or
R10 together with R2 represents a a C₃₋₇ cycloalkyl group;
m is zero or an integer from 1 to 3; n is zero or an integer from 1 to 3; both p and r are independently zero or an integer from 1 to 4; q is an integer from 1 to 4;
provided that , when R1 and R2 together with nitrogen and carbon atom to which they are attached respectively represent a 5 to 6 membered heterocyclic group, i) m is 1 or 2; ii) when m is 1, R is not fluorine and iii) when m is 2, the two substituents R are not both fluorine,
and pharmaceutically acceptable salts and solvates thereof.

16. The method according to claim 10, wherein the NK1 receptor antagonist comprises a compound of formula (II): 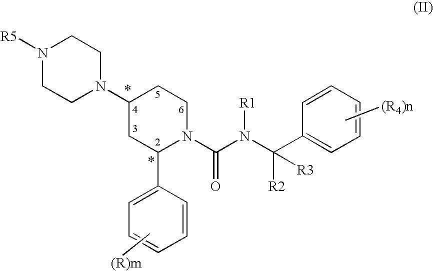
wherein,
R represents a halogen atom or a C₁₋₄ alkyl group;
R₁ represents a C₁₋₄ alkyl group;
R₂ represents hydrogen or a C₁₋₄ alkyl group;
R₃ represents hydrogen or C₁₋₄ alkyl group;
R₄ represents a trifluoromethyl group;
R₅ represents hydrogen, a C₁₋₄ alkyl group or C(O)R₆;
R₆ represents C₁₋₄ alkyl, C₃₋₇ cycloalkyl, NH(C₁₋₄ alkyl) or N(C₁₋₄alkyl)₂;
m is zero or an integer from 1 to 3;
n is an integer from 1 to 3;
and pharmaceutically acceptable salts and solvates thereof.

17. The method according to claim 10, wherein the NK1 receptor antagonist comprises a compound of formula (III): 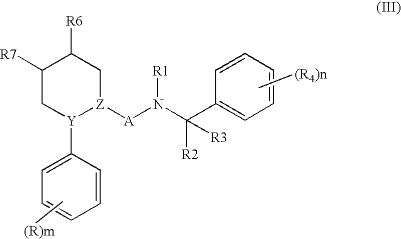
wherein,
R represents halogen or C₁₋₄ alkyl;
R₁ represents C₁₋₄ alkyl;
R₂ or R₃ independently represent hydrogen or C₁₋₄ alkyl;
R₄ represents trifluoromethyl, C₁₋₄ alkyl, C₁₋₄ alkoxy, trifluoromethoxy or halogen;
R₅ represents hydrogen , C₁₋₄ alkyl or C₃₋₇ cycloalkyl;
R₆ is hydrogen and R₇ is a radical of formula (W): 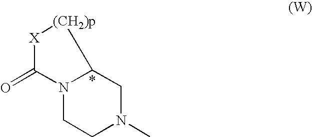
or R₆ is a radical of formula (W) and R₇ is hydrogen;
X represents CH₂, NR₅ or O;
Y represents Nitrogen and Z is CH or Y represents CH and Z is Nitrogen;
A represents C(O) or S(O)q, provided that when Y is nitrogen and Z is CH, A is not S(O)q;
m is zero or an integer from 1 to 3;
n is an integer from 1 to 3;
p and q are independently an integer from 1 to 2;
and pharmaceutically acceptable salts and solvates thereof.

18. The method according to claim 10, wherein the NK1 receptor antagonist is selected from the group consisting of 2-(S)-(4-fluoro-2-methyl-phenyl)-piperazine-1-carboxylic acid [1-(R)-(3,5-bis-trifluoromethyl-phenyl)-ethyl]-methyl-amide or pharmaceutically acceptable salts or solvates thereof, 4-(S)-(4-acetyl-piperazin-1-yl)-2-(R)-(4-fluoro-2-methyl-phenyl)-piperidine-1-carboxylic acid [1-(R)-(3,5-bis-trifluoromethyl-phenyl)-ethyl]-methylamide or pharmaceutically acceptable salts or solvates thereof, and 2-(R)-(4-fluoro-2-methyl-phenyl)-4-(S)-((8aS)-6-oxo-hexahydro-pyrrolo[1,2-a]-pyrazin-2-yl)-piperidine-1-carboxylic acid [1-(R)-(3,5-bis-trifluoromethyl-phenyl)-ethyl]-methylamide or pharmaceutically acceptable salts or solvates thereof.

19. The method according to Caim 10, wherein the selective serotonin reuptake inhibitor is paroxetine.

20. The method according to claim 10, wherein the NK1 receptor antagonist is 2-(S)-(4-fluoro-2-methyl-phenyl)-piperazine-1-carboxylic acid [1-(R)-(3,5-bis-trifluoromethyl-phenyl)-ethyl]-methyl-amide or pharmaceutically acceptable salts or solvates thereof and the selective serotonin reuptake inhibitor is paroxetine.

21. The method according to claim 10, wherein the NK1 receptor antagonist is 4-(S)-(4-acetyl-piperazin-1-yl)-2-(R)-(4-fluoro-2-methyl-phenyl)-piperidine-1-carboxylic acid [1-(R)-(3,5-bis-trifluoromethyl-phenyl)-ethyl]-methylamide or pharmaceutically acceptable salts or solvates thereof and the selective serotonin reuptake inhibitor is paroxetine.

22. The method according to claim 10, wherein the NK1 receptor antagonist is 2-(R)-(4-fluoro-2-methyl-phenyl)-4-(S)-((8aS)-6-oxo-hexahydro-pyrrolo[1,2-a]-pyrazin-2-yl)-piperidine-1-carboxylic acid [1-(R)-(3,5-bis-trifluoromethyl-phenyl)-ethyl]-methylamide or pharmaceutically acceptable salts or solvates thereof and the selective serotonin reuptake inhibitor is paroxetine.

23. A composition comprising an NK1 receptor antagonist and a selective serotonin reuptake inhibitor.

24. A pharmaceutical formulation comprising an effective amount of an NK1 receptor antagonist, an effective amount of a selective serotonin reuptake inhibitor, and a pharmaceutically acceptable excipient.
